(19) United States
(12) Patent Application Publication
(10) Pub. No.: US 2008/0221120 A1
(43) Pub. Date: Sep. 11, 2008

(54) Modulation of Aquaporin in Modulation of Angiogenesis and Cell Migration

(76) Inventor: Alan Verkman, San Francisco, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/908,399

(22) PCT Filed: Mar. 21, 2006
(86) PCT No.: PCT/US06/10518

§ 371 Date: Apr. 8, 2008

Related U.S. Application Data
(60) Provisional application No. 60/664,801, filed on Mar. 23, 2005.

(51) Int. Cl. A61K 31/496; A61K 31/495; A61K 31/4152; C12Q 1/18; A61P 7/00

(52) U.S. Cl. 514/253.06; 514/404; 514/252.12; 435/32

(57) ABSTRACT

The invention provides compositions, pharmaceutical preparations, and methods for modulating angiogenesis and/or cell migration in a subject having a cellular proliferative disease (e.g., cancer), or a disease or condition amenable to treatment by enhancing cellular proliferation and cell migration (e.g., angiogenesis), by modulating the activity of an aquaporin, such as aquaporin-1. The compositions and pharmaceutical preparations of the invention may comprise one or more of compounds that modulate the activity of aquaporin-1.

### CROSS-REFERENCE TO RELATED APPLICATIONS

This application claims the priority benefit of U.S. provisional application Ser. No. 60/664,801, filed Mar. 23, 2005, which application is incorporated herein by reference in its entirety.

### STATEMENT REGARDING FEDERALLY SPONSORED RESEARCH

This invention was made with government support under grant no. DK35124 awarded by the National Institutes of Health. The government may have certain rights in this invention.

### BACKGROUND OF THE INVENTION

Aquaporin-1 (AQP1, also known as Channel-Like Integral Membrane Protein, 28-KD (CHIP28), and Aquaporin-CHIP (AQP1)) is a 28-kD water channel protein originally isolated from the plasma membranes of red blood cells and renal tubules by Denker et al. J. Biol. Chem. 263: 15634-15642 (1988). The polypeptide structure of AQP1 contains 6 bilayer-spanning domains, 2 exofacial potential N-glycosylation sites, and intracellular N and C termini (Preston et al. Proc. Nat. Acad. Sci. 88: 11110-11114 (1991)). APQ1 has strong homology with the major intrinsic protein of bovine lens (MIP26), which is now referred to as aquaporin 0 (AQP0) and is considered the prototype of an ancient family of membrane channels. AQP1 has been shown to act as a 2-stage filter where the conserved NPA (asp-pro-ala) motifs form a selectivity-determining, or size-exclusion, region (de Groot et al., Science 294: 2353-2357 (2001)).

The atomic structure of mammalian AQP1 illustrates how this family of proteins is freely permeated by water but not protons (hydronium ions, H(3)O+). The mercury sensitivity of AQP1 is well explained by localization of the specific residue (C189) at the narrowest segment of the channel at the same level as H180 and R195. Cysteines are present at the corresponding position in several other members of the aquaporin family (AQP2, 107777; AQP5, 600442; AQP6, 601383; and AQP9, 602914).

The atomic model of AQP1 at 3.8 angstrom was first described by Murata et al. (Nature 407: 599-605 (2000)) from electron crystallographic data. The structure revealed that multiple highly conserved amino acid residues stabilize the novel fold of AQP1. In addition, the analysis further revealed that the aqueous pathway is lined with conserved hydrophobic residues that permit rapid water transport, whereas the water selectivity is due to a constriction of the pore diameter to about 3 angstroms over a span of 1 residue. The atomic model provided a possible molecular explanation to a longstanding puzzle in physiology—how membranes can be freely permeable to water but impermeable to protons.

The atomic model of AQP1 at 2.2 angstrom was later described by Sui et al. (Nature 414: 872-878 (2001)). The analysis showed that the channel consists of 3 topologic elements, an extracellular and a cytoplasmic vestibule connected by an extended narrow pore or selectivity filter, averaging 4 angstroms in diameter. Within the selectivity filter, 4 bound waters are localized along 3 hydrophilic nodes, which punctuate an otherwise extremely hydrophobic pore segment, facilitating water transport. The highly conserved histidine-182 residue is critical in establishing water specificity. AQP1 lacks a suitable chain of hydrogen-bonded water molecules in the selectivity filter that could act as a proton wire, indicating that proton transport through the channel is highly energetically unfavorable. Two other related proteins were found to be water transporters and are members of the aquaporin family (Fushimi et al. Nature 361: 549-552 (1993); Maurel et al. EMBO J. 12: 2241-2247 (1993)).

The role of AQP1 in physiology has not been well defined. AQP1 is expressed in diverse epithelia with distinct developmental patterns. By immunochemical and functional means, Smith et al. (J. Clin. Invest. 92: 2035-2041 (1993)) showed that AQP1 is essentially absent in neonatal red cells of the rat. After birth, AQP1 appears in the red cells and increases within several weeks to the adult level of expression. The neonatal kidney, while displaying low levels of AQP1 expression, has a parallel increase in the amount and distribution of AQP1 in the proximal tubules and the descending thin limbs of the loops of Henle, commensurate with the kidney's ability to form concentrated urine. It was further suggested that the water channels act to promote the rehydration of red cells after their shrinkage in the hypertonic environment of the renal medulla. Rapid rehydration would return the cells to their normal volume, optimizing their deformability for transit in the microcirculation (Hoffman et al., J. Clin. Invest. 92: 1604-1605 ((1993)).

Analysis of AQP1 defective individuals has not identified any serious detriment associated with decreased function of AQP1. King et al. (New Eng. J. Med. 345: 175-179 (2001)) found that AQP1 deficiency resulted in defective urinary concentrating ability when the subjects were water deprived. Preston et al. (Science 265: 1585-1587 (1994)) reported that individuals having an AQP1 gene defect suffered no apparent clinical consequence, despite that two of the individuals were homozygous for different nonsense mutations (exon deletion or frameshift), and a third had a missense mutation encoding a nonfunctioning CHIP molecule. AQP1 has also been suggested to have a role in vascular permeability in the lung. (King et al., 2001).

AQP1 is expressed widely in vascular endothelia where it increases cell membrane water permeability (Nielsen et al. Proc Natl Acad Sci USA 90, 7275-9 (1993); Carter et al. Biophys J 74, 2121-8 (1998); Hasegawa et al. Am J Physiol 266, C893-903 (1994)). However, the role of AQP1 in endothelial cell function was previously unknown. AQP1 protein is expressed strongly in proliferating microvessels in human (Saadoun et al. Br J Cancer 87, 621-3 (2002)) and rat (Endo et al. Microvasc Res 58, 89-98 (1999)) malignant brain tumors, bone marrow microvessels in human multiple myeloma (Vacca et al. Br J Haematol 113, 415-21 (2001)), and proliferating microvessels in chick embryo chorioallantoic membrane (Ribatti et al. Anat Rec 268, 85-9 (2002).). Thus, indirect evidence supports a role for AQP1 in microvessel formation/function. Increased aquaporin expression has also been observed in malignant tumor cells (Saadoun et al. Br J Cancer 87, 621-3 (2002); Moon et al. Oncogene 22, 6699-703 (2003)) suggesting a role for water channels in tumor growth/spread. Again, these studies have not offered any clinically useful therapies.

Identification of agents that act to inhibit tumor cell growth, but which do not substantially affect normal (non-cancerous) cells has been hampered by the lack of a target for therapy that is not common to both tumor and normal cells, or for which function in normal cells is critical. There is accordingly still a need for new methods for modulation of angiogenesis and cell migration with respect to cellular proliferative diseases. The present invention addresses these needs, as well as others.

### SUMMARY OF THE INVENTION

The invention provides compositions, pharmaceutical preparations, and methods for modulating angiogenesis and/or cell migration in a subject having a cellular proliferative disease (e.g., cancer), or a disease or condition amenable to treatment by enhancing cellular proliferation and cell migration (e.g., angiogenesis), by modulating the activity of an aquaporin, such as aquaporin-1. The compositions and pharmaceutical preparations of the invention may comprise one or more of compounds that modulate the activity of aquaporin-1.

In one aspect, the present invention provides a method of modulating angiogenesis in a subject, comprising administering the subject an agent that modulates a biological activity of an aquaporin-1 (AQP1). In one embodiment, agent inhibits the biological activity of the APQ1. In another embodiment, the agent enhances the biological activity of the AQP1. In some embodiments, the subject is a mammal, such as a human. In some embodiments, the administering provides for inhibition of angiogenesis in a tumor. In other embodiments, the administering provides for enhancement of wound healing.

In another aspect, the present invention provides a method for treating a cellular proliferative disease in a subject, comprising administering the subject an agent that modulates a biological activity of an aquaporin-1 (AQP1). In some embodiments, the cellular proliferative disease is cancer. In some embodiments, the agent inhibits the biological activity of an aquaporin. In some embodiments, the aquaporin is aquaporin-1. In further embodiments, the subject is a human.

In another aspect, the present invention provides a method of identifying an agent that modulates activity of aquaporin-1 including, culturing a cell expressing an aquaporin-1 (AQP1) in the presence of an agent, and determining the effect of the agent on at least one of cell migration, cell adhesion, or cell proliferation, wherein a change in one of cell migration, cell adhesion, and cell proliferation in the presence of the agent as compared to the absence of the agent indicates the agent modulates the activity of AQP1. In some embodiments, the cell is a mammalian cell. In further embodiments, the AQP1 is recombinantly expressed in the cell. In some embodiments, an increase in one of cell migration, cell adhesion, and cell proliferation, indicates the agent increases activity of the aquaporin. In other embodiments, a decrease in one of cell migration, cell adhesion, and cell proliferation, indicates the agent increases activity of the aquaporin.

In yet another aspect, the present invention provides, a pharmaceutical composition comprising a compound of formula (I):

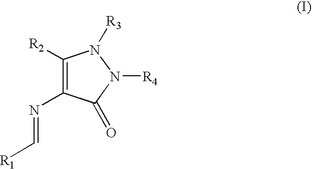

wherein R₁ is independently selected from a substituted or unsubstituted phenyl group; R₂ is independently chosen from a hydrogen, or an allyl group; R₃ is independently chosen from a hydrogen, or an alkyl group; and R₄ is independently selected from a substituted or unsubstituted phenyl group; or a pharmaceutically acceptable derivative thereof, as an individual stereoisomer or a mixture thereof. In some embodiments, the composition further comprises at least one of a pharmaceutically acceptable carrier, a pharmaceutically acceptable diluent, a pharmaceutically acceptable excipient and a pharmaceutically acceptable adjuvant.

In one embodiment, R₁ is a 2-(nitro)-4-(bromo)-5-(hydroxy)phenyl group. In another embodiment, R₂ is a methyl group. In yet another embodiment, R₃ is a methyl group. In yet another embodiment, R₄ is an unsubstituted phenyl group. In some embodiments, the compound is:

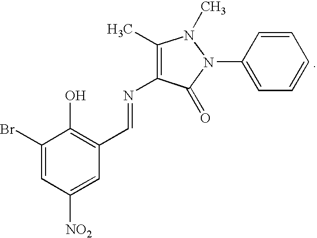

In yet another aspect, the present invention provides a pharmaceutical composition comprising a compound of formula (II):

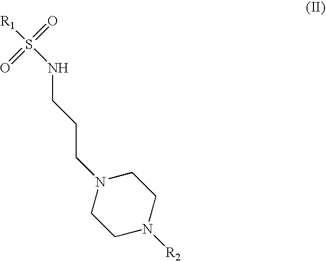

wherein R₁ is independently selected from a substituted or unsubstituted phenyl group, or a substituted or unsubstituted heteroaromatic group; and R₂ is independently selected from a substituted or unsubstituted phenyl group; or a pharmaceutically acceptable derivative thereof, as an individual stereoisomer or a mixture thereof. In some embodiments, the composition further comprises at least one of a pharmaceutically acceptable carrier, a pharmaceutically acceptable diluent, a pharmaceutically acceptable excipient and a pharmaceutically acceptable adjuvant. In one embodiment, R₁ is a unsubstituted quinolinyl group. In another embodiment, R₂ is a 2-(fluoro)phenyl group. In some embodiments the compound is:

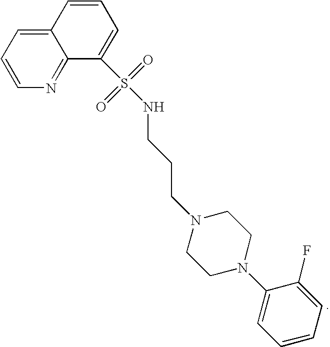

These and other objects and advantages of the invention will be apparent from the detailed description below.

### BRIEF DESCRIPTION OF THE DRAWINGS

The invention will be more fully understood by reference to the following drawings, which are for illustrative purposes only.

FIG. 1 shows reduced tumor growth in AQP1 null mice. Panel A shows the growth of subcutaneous melanoma in wildtype (AQP1^{+/+}), AQP1 null (AQP1^{-/-}), and AQP3 null (AQP3^{-/-}) mice (n=5-10, p<0.001 comparing AQP1^{+/+} vs. AQP1^{-/-} or AQP3^{-/-} vs. AQP1^{-/-}). Panel B shows the survival rates of wildtype (AQP1^{+/+}) and AQP1 null (AQP1^{-/-}) mice with subcutaneous melanoma (n=9-10, p<0.001). Panel C shows growth of subcutaneous melanoma in C57/BL6 mice (n=5 each, p<0.001). Panel D shows the volume of brain melanoma (n=5-7) of wildtype (AQP1^{+/+}) and AQP1 null (AQP1^{-/-}) mice.

FIG. 2, Panel A shows tumors from wildtype (AQP1^{+/+}) and AQP1 null (AQP1^{-/-}) mice stained with isolectin-B4 (brown). The inserts show tumor vessels immunostained for AQP1. Panel B shows the number of vessels (left), percent of necrotic area (center), and number of islands in subcutaneous melanoma (right) of wildtype (AQP1^{+/+}) and AQP1 null (AQP1^{-/-}) mice (Mean ±SEM, 6 mice per group, *p<0.05, **p<0.01, ***p<0.001).

FIG. 3 shows Angiogenesis in Matrigel and characterization of cultured endothelial cells. (left) Representative Matrigel pellets. (center) Increased vascularity (H&E stain) and (right) higher hemoglobin content of bFGF-supplemented Matrigel from AQP1^{+/+} vs. AQP1^{-/-} mice (n=8-9). (Inserts) Vessels in Matrigel (arrowheads) immunostained for AQP1.

FIG. 4 shows (left panel) phase contrast micrograph of cultured endothelial cells. The right panel shows proliferation of AQP1^{+/+} and AQP1^{-/-} endothelial cells over 4 days, with times points taken at zero, 2, and 4 days (n=12 each, differences not significant).

FIG. 5 shows (left panel) AQP1 immunostaining (red) with DAPI counterstain (blue) of wildtype (AQP1^{+/+}) and AQP1 null (AQP1^{-/-}) mice. The right panel shows plasma membrane water permeability measured by calcein fluorescence quenching in response to osmotic gradients. (Mean ±SEM, *p<0.05).

FIG. 6 provides results of experiments showing impaired migration of endothelial cells lacking AQP1. Panel A shows in the left panel adhesion of endothelial cells (blue) on transwell filters (pre-scraping) and in the right panel quantification of percent of total adherent cells (n=9-10 cultures, difference not significant). Panel B shows in the left panel migrated endothelial cells (post-scraping) and in the right panel summary of the migration data (n=16-20). Panel C shows in the left panel endothelial network of ‘cords’ of endothelial cells expressing AQP1 or lacking AQP1, and in the right panel shows a summary of the numbers of cords of indicated diameters (n=11-18) for each class. Panel D shows wound healing of cultured endothelial cells in the left panel visualized using phase contrast (initial wound edge blue, after 24 h red) and quantified in the right panel as the wound edge speed (n=4 per group). (Mean ±SEM, *p<0.05, **p<0.01, ***p<0.001).

FIG. 7, Panel A shows AQP1 and AQP4 immunostaining (red) in control and transfected cells for CHO cells (left) and FRT cells (right). Panel B shows results of cell proliferation assays (left), adhesion assays (middle), and migration assays (right) for CHO and FRT cells expressing either AQP1 or AQP4 (n=8-12).

FIG. 8, Panel A shows micrographs wound healing (left) with data summary (center) (n=8-12) for CHO and FRT cells expressing either AQP1 or AQP4, and tracks (right) of migrating control CHO cells and CHO cells expressing AQP1 over 4 hours. Arrows indicate initial positions. Panel B shows membrane ruffles (arrowheads) in control CHO cells and CHO cells expressing AQP1 (left), the mean number of ruffles (8 migrating cells per group, over 4 h) (center), and a immunostain (right) showing AQP1 polarizes to lamellipodia in migrating CHO cells (Mean ±SEM, *p<0.05, **p<0.01, ***p<0.001).

FIG. 9 shows a series of images of control and AQP1-expressing tumor cells. Panel A shows phase-contrast micrographs of control and AQP1-expressing tumor cells (Scale bar, 50 μm). Panel B shows AQP1 immunostaining (red) with blue nuclear stain (Scale bar, 25 μm).

FIG. 10, Panel A, shows osmotically-driven cell plasma membrane water permeability as measured by calcein fluorescence quenching. In these experiments perfusate solutions changed rapidly between osmolalities of 600 and 300 mosM. Panel B shows a summary of the osmotic equilibration rates 1/t (SE, n=4-6 per condition, * P<0.01).

FIG. 11, Panel A shows cell proliferation as measured by ³H-thymidine incorporation in B16F10 and 4T1 cell lines either expressing or not expressing AQP1. Panel B shows growth curves obtained by cell counting for 4T1 (top) and B16F10 (bottom) cell lines either expressing (closed circles) or not expressing (open circles) AQP1.

FIG. 12, Panel A, shows cell migration measured using the modified Boyden chamber (transwell) migration assay. Coomassie blue staining before (left) and after (right) scraping non-migrated cells on the upper surface of the porous membrane. Panels B and C shows a summary of percent migration at 6 hours in three independent sets of experiments for the 4T1 cells (Panel B) and B16F10 cells (Panel C), in 3-6 wells for each cell lines (SE, P<0.05). Where indicated, cells were transfected with YFP plasmid (“mock”) or fluorescently labeled prior to migration assay (“CMRDA” or “CMFDA”).

FIG. 13, Panel A shows cell adherence to type I collagen, fibronectin and laminin, expressed as percentage compared to BSA control (SE, n=6, differences not significant) for either 4T1 cells (left) or B16F10 cells (right). Panel B shows cell invasion measured by transwell assay with matrigel-coated porous membrane at 15 hours (SE, n=3, *P<0.05) for either 4T1 cells (top) or B16F10 cells (bottom).

FIG. 14 shows results of wound healing assays of tumor cell migration. Panel A shows phase-contrast micrographs of scratched monolayers of control and AQP 1-expressing B16F10 cells at 0 and 15 hours (Scale bar, 100 μm). Panel B shows a summary of wound healing at 15 hours after scratch (SE, n=6, * P<0.05, ** P<0.01). Panel C shows AQP1 immunofluorescence (red) at the leading edge of migrating AQP1-expressing B16F10 cells during wound closure. Arrows indicate AQP1 expression in lamellipodia. Scale bar, 20 μm. Panel D shows phase-contrast micrographs (top of the leading edge of AQP1-expressing B16F10 cells lamella (arrows, Bar: 25 μm) and summary data (bottom) of the lamella width at the leading edge of wound (SE, n=5-6, * P<0.05).

FIG. 15, Panel A shows increase lung extravasation of AQP1-expressing tumor cells after tail vein injection. Control (CMRA labeled, red fluorescent) and AQP1-expressing 4T1 cells (CMFDA labeled, green fluorescent) were mixed 1:1 and applied to transwell filters. The upper chamber contained 1% serum and the lower chamber contained 10% serum. Fluorescence micrographs showing red and green cells before (left) and after (middle) scraping non-migrated cells from the upper surface of the porous membrane. Scale bar, 50 μm. Panel B shows a summary of ratios of AQP1-expressing vs. control cells that migrated in 6 hours (SD, n=6, * P<0.05).

FIG. 16, Panel A shows fluorescence micrograph of red CMRA-labeled AQP1-expressing 4T1 cells mixed 1:1 with green CMFDA-labeled control 4T1 cells (Scale bar, 200 μm). Paneled B and C show frozen sections of mouse lung at 6 hours after tail vein injection of 1:1 cell mixtures with indicated red/green labeling (Scale bar, 50 μm).

FIG. 17 shows summaries of tumor cell count ratios (green/red or red/green cells as indicated) and number of cells per microscope field in the injection mixture (open bars) and in lung at 10 min and 6 hours after tail vein injection (SD, n=3 for 10 min group, n=6-8 for 6 hours group, * P<0.05). Labeling scheme shown at the bottom, with experiments done for CMRA labeling of control and CMFDA labeling of AQP1-expressing cells, and with the labeling reversed (CMFDA labeling of control and CMRA labeling of AQP1-expressing cells).

FIG. 18, Panel A, left and middle, show hematoxylin and eosin-staining of paraformaldehye-fixed paraffin-embedded sections of mouse lung tissue at 14 days after tail vein injection of 10⁶ control or AQP1-expressing 4T1 cells. Tumor metastases are indicated by arrows. The right panel shows AQP1 immunohistochemistry showing labeling (brown) of alveoli/vessels in both micrographs, with AQP1 also in tumor cells in the lower micrograph. Panel B is a summary of data showing number of metastases per lung, area of tumor colonies, and alveolar wall thickness within 50 μm of metastases (SE, 5 mice per group, * P<0.02).

FIG. 19, Panel A is a series of graphs showing tumor size at 15 and 18 days after subcutaneous implantation of 10⁶ B16F10 (right) or 2×10⁵ 4T1 cells (left) (control and AQP1-expressing) (SE, 20 mice per group, differences not significant). Panel B shows histology of peripheral tumors at 15 days after subcutaneous implantation of control or AQP1-expressing 4T1 cells.

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It should be noted that, as used herein and in the appended claims, the singular forms “a”, “and”, and “the” include plural referents unless the context clearly dictates otherwise. Thus, for example, reference to “a compound” includes a plurality of such compounds, and reference to “the cell” includes reference to one or more cells and equivalents thereof known to those skilled in the art, and so forth.

The publications discussed herein are provided solely for their disclosure prior to the filing date of the present application, and are incorporated herein by reference. Nothing herein is to be construed as an admission that the present invention is not entitled to antedate such publication by virtue of prior invention. Further, the dates of publication provided may be different from the actual publication dates that may need to be independently confirmed.

The definitions used herein are provided for reason of clarity, and should not be considered as limiting. The technical and scientific terms used herein are intended to have the same meaning as commonly understood by those of ordinary skill in the art to which the invention pertains.

### DETAILED DESCRIPTION OF THE INVENTION

The present invention provides compositions, pharmaceutical preparations, and methods for modulating angiogenesis and/or cell migration in a subject having a cellular proliferative disease (e.g., cancer) by modulating the activity of an aquaporin, such as aquaporin-1. The compositions and pharmaceutical preparations of the invention may comprise one or more of compounds that modulate the activity of aquaporin-1.

### DEFINITIONS

As used herein “Aquaporin-1”, “AQP1”, “Channel-Like Integral Membrane Protein, 28-KD”, “CHIP28”, and “Aquaporin-CHIP” refer to a 28-kD water channel protein originally isolated from the plasma membranes of red blood cells and renal tubules and described in Denker et al. J. Biol. Chem. 263: 15634-15642 (1988). The aquaporins are a family of intrinsic membrane proteins that function as water-selective channels in the plasma membranes of the cells of many water transporting tissues.

As used herein “angiogenesis” refers to the process of growing blood vessels from endothelial cells which results in, among other characteristics, the vascularization of tissue. Under normal physiological conditions, angiogenesis occurs under particular conditions such as in wound healing, during tissue and organ regeneration, during embryonic vasculature development, as well as in the formation of the corpus luteum, endometrium, and placenta. Excessive angiogenesis, however, has been associated with a number of disease conditions. Examples of diseases associated with excessive angiogenesis include rheumatoid arthritis, atherosclerosis, diabetes mellitus, retinopathies, psoriasis, and retrolental fibroplasia. In addition, angiogenesis has been identified as a critical requirement for solid tumor growth and cancer metastasis. Examples of tumor types associated with angiogensis include rhabdomyosarcomas, retinoblastoma, Ewing's sarcoma, neuroblastoma, osteosarcoma, hemangioma, leukemias, and neoplastic diseases of the bone marrow involving excessive proliferation of white blood cells. Due to the association between angiogenesis and various disease conditions, substances that have the ability to modulate angiogenesis would be potentially useful treatments for these disease conditions.

“Cellular proliferative disease” as used herein refers to any condition characterized by the undesired propagation of cells, including, but not limited to, neoplastic disease conditions, e.g., cancer. Examples of cellular proliferative disease include, but not limited to, abnormal stimulation of endothelial cells (e.g., atherosclerosis), solid tumors and tumor metastasis, benign tumors, for example, hemangiomas, acoustic neuromas, neurofibromas, trachomas, and pyogenic granulomas, vascular malfunctions, abnormal wound healing, inflammatory and immune disorders, Bechet's disease, gout or gouty arthritis, abnormal angiogenesis accompanying, for example, rheumatoid arthritis, psoriasis, diabetic retinopathy, other ocular angiogenic diseases such as retinopathy of prematurity (retrolental fibroplastic), macular degeneration, corneal graft rejection, neurovascular glaucoma and Oster Webber syndrome, psoriasis, restinosis, fungal, parasitic and viral infections such cytomegaloviral infections.

The term “modulates” as used here refers to an increase or a decrease in activity. In some embodiments, the modulation is at least 10%, at least 20%, at least 30%, at least 40%, at least 50%, at least 60%, at least 70%, at least 80%, at least 90%, at least 95%, or at least 100%.

“In combination with” as used herein refers to uses where, for example, the first compound is administered during the entire course of administration of the second compound; where the first compound is administered for a period of time that is overlapping with the administration of the second compound, e.g. where administration of the first compound begins before the administration of the second compound and the administration of the first compound ends before the administration of the second compound ends; where the administration of the second compound begins before the administration of the first compound and the administration of the second compound ends before the administration of the first compound ends; where the administration of the first compound begins before administration of the second compound begins and the administration of the second compound ends before the administration of the first compound ends; where the administration of the second compound begins before administration of the first compound begins and the administration of the first compound ends before the administration of the second compound ends. As such, “in combination” can also refer to regimen involving administration of two or more compounds. “In combination with” as used herein also refers to administration of two or more compounds which may be administered in the same or different formulations, by the same of different routes, and in the same or different dosage form type.

The term “isolated compound” means a compound which has been substantially separated from, or enriched relative to, other compounds with which it occurs in nature. Isolated compounds are usually at least about 80%, more usually at least 90% pure, even more preferably at least 98% pure, most preferably at least about 99% pure, by weight. The compounds considered within the scope of the present disclosure include diastereomers as well as their racemic and resolved, ehantiomerically pure forms and pharmaceutically acceptable salts thereof.

“Treating” or “treatment” of a condition or disease includes: (1) preventing at least one symptom of the conditions, i.e., causing a clinical symptom to not significantly develop in a mammal that may be exposed to or predisposed to the disease but does not yet experience or display symptoms of the disease, (2) inhibiting the disease, i.e., arresting or reducing the development of the disease or its symptoms, or (3) relieving the disease, i.e., causing regression of the disease or its clinical symptoms. As used herein, the term “treating” is thus used to refer to both prevention of disease, and treatment of pre-existing conditions. For example, where an AQP1 inhibitor is administered, the prevention of cellular proliferation can be accomplished by administration of the subject compounds prior to development of overt disease, e.g. to prevent the regrowth of tumors, prevent metastatic growth, etc. Alternatively the compounds are used to treat ongoing disease, by stabilizing or improving the clinical symptoms of the patient.

A “therapeutically effective amount” or “efficacious amount” means the amount of a compound that, when administered to a mammal or other subject for treating a disease, is sufficient to effect such treatment for the disease. The “therapeutically effective amount” will vary depending on the compound, the disease and its severity and the age, weight, etc., of the subject to be treated.

The terms “subject” and “patient” mean a member or members of any mammalian or non-mammalian species that may have a need for the pharmaceutical methods, compositions and treatments described herein. Subjects and patients thus include, without limitation, primate (including humans), canine, feline, ungulate (e.g., equine, bovine, swine (e.g., pig)), avian, and other subjects. Humans and non-human animals having commercial importance (e.g., livestock and domesticated animals) are of particular interest.

“Mammal” means a member or members of any mammalian species, and includes, by way of example, canines; felines; equines; bovines; ovines; rodentia, etc. and primates, particularly humans. Non-human animal models, particularly mammals, e.g. primate, murine, lagomorpha, etc. may be used for experimental investigations.

The term “unit dosage form,” as used herein, refers to physically discrete units suitable as unitary dosages for human and animal subjects, each unit containing a predetermined quantity of a compound of interest calculated in an amount sufficient to produce the desired effect in association with a pharmaceutically acceptable diluent, carrier or vehicle. The specifications for the novel unit dosage forms of compounds disclosed herein depend on the particular compound employed and the effect to be achieved, and the pharmacodynamics associated with each compound in the host.

The term “physiological conditions” is meant to encompass those conditions compatible with living cells, e.g., predominantly aqueous conditions of a temperature, pH, salinity, etc. that are compatible with living cells.

A “pharmaceutically acceptable excipient,” “pharmaceutically acceptable diluent,” “pharmaceutically acceptable carrier,” and “pharmaceutically acceptable adjuvant” means an excipient, diluent, carrier, and adjuvant that are useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. “A pharmaceutically acceptable excipient, diluent, carrier and adjuvant” as used in the specification and claims includes both one and more than one such excipient, diluent, carrier, and adjuvant.

As used herein, a “pharmaceutical composition” is meant to encompass a composition suitable for administration to a subject, such as a mammal, especially a human. In general a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intracheal and the like. In some embodiments the composition is suitable for administration by a transdermal route, using a penetration enhancer other than DMSO. In other embodiments, the pharmaceutical compositions are suitable for administration by a route other than transdermal administration.

As used herein, “pharmaceutically acceptable derivatives” of a compound is meant to include salts, esters, enol ethers, enol esters, acetals, ketals, orthoesters, hemiacetals, hemiketals, acids, bases, solvates, hydrates or prodrugs thereof. Such derivatives may be readily prepared by those of skill in this art using known methods for such derivatization.

The compounds produced may be administered to animals or humans without substantial toxic effects and either are pharmaceutically active or are prodrugs.

A “pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

A “pharmaceutically acceptable ester” of a compound means an ester that is pharmaceutically acceptable and that possesses the desired pharmacological activity of the parent compound, and includes, but is not limited to, alkyl, alkenyl, alkynyl, aryl, heteroaryl, aralkyl, heteroaralkyl, cycloalkyl and heterocyclyl esters of acidic groups, including, but not limited to, carboxylic acids, phosphoric acids, phosphinic acids, sulfonic acids, sulfinic acids and boronic acids.

A “pharmaceutically acceptable enol ether” of a compound means an enol ether that is pharmaceutically acceptable and that possesses the desired pharmacological activity of the parent compound, and includes, but is not limited to, derivatives of formula C═C(OR) where R is hydrogen, alkyl, alkenyl, alkynyl, aryl, heteroaryl, aralkyl, heteroaralkyl, cycloalkyl or heterocyclyl.

A “pharmaceutically acceptable enol ester” of a compound means an enol ester that is pharmaceutically acceptable and that possesses the desired pharmacological activity of the parent compound, and includes, but is not limited to, derivatives of formula C═C(OC(O)R) where R is hydrogen, alkyl, alkenyl, alkynyl, aryl, heteroaryl, aralkyl, heteroaralkyl, cycloalkyl or heterocyclyl.

A “pharmaceutically acceptable solvate or hydrate” of a compound means a solvate or hydrate complex that is pharmaceutically acceptable and that possesses the desired pharmacological activity of the parent compound, and includes, but is not limited to, complexes of a compound disclosed herein with one or more solvent or water molecules, or 1 to about 100, or 1 to about 10, or one to about 2, 3 or 4, solvent or water molecules.

“Pro-drugs” means any compound that releases an active parent drug according to formula (I) in vivo when such prodrug is administered to a mammalian subject. Prodrugs of a compound of formula (I) are prepared by modifying functional groups present in the compound of formula (I) in such a way that the modifications may be cleaved in vivo to release the parent compound. Prodrugs include compounds of formula (I) wherein a hydroxy, amino, or sulfhydryl group in compound (I) is bonded to any group that may be cleaved in vivo to regenerate the free hydroxyl, amino, or sulfhydryl group, respectively. Examples of prodrugs include, but are not limited to esters (e.g., acetate, formate, and benzoate derivatives), carbamates (e.g., N,N-dimethylaminocarbonyl) of hydroxy functional groups in compounds of formula (I), and the like.

The term “organic group” and “organic radical” as used herein means any carbon-containing group, including hydrocarbon groups that are classified as an aliphatic group, cyclic group, aromatic group, functionalized derivatives thereof and/or various combinations thereof. The term “aliphatic group” means a saturated or unsaturated linear or branched hydrocarbon group and encompasses alkyl, alkenyl, and alkynyl groups, for example. The term “alkyl group” means a substituted or unsubstituted, saturated linear or branched hydrocarbon group or chain (e.g., C₁ to C₈) including, for example, methyl, ethyl, isopropyl, tert-butyl, heptyl, iso-propyl, n-octyl, dodecyl, octadecyl, amyl, 2-ethylhexyl, and the like. Suitable substituents include carboxy, protected carboxy, amino, protected amino, halo, hydroxy, protected hydroxy, nitro, cyano, monosubstituted amino, protected monosubstituted amino, disubstituted amino, C₁ to C₇ alkoxy, C₁ to C₇ acyl, C₁ to C₇ acyloxy, and the like. The term “substituted alkyl” means the above defined alkyl group substituted from one to three times by a hydroxy, protected hydroxy, amino, protected amino, cyano, halo, trifloromethyl, mono-substituted amino, di-substituted amino, lower alkoxy, lower alkylthio, carboxy, protected carboxy, or a carboxy, amino, and/or hydroxy salt. As used in conjunction with the substituents for the heteroaryl rings, the terms “substituted (cycloalkyl)allyl” and “substituted cycloalkyl” are as defined below substituted with the same groups as listed for a “substituted alkyl” group. The term “alkenyl group” means an unsaturated, linear or branched hydrocarbon group with one or more carbon-carbon double bonds, such as a vinyl group. The term “alkynyl group” means an unsaturated, linear or branched hydrocarbon group with one or more carbon-carbon triple bonds. The term “cyclic group” means a closed ring hydrocarbon group that is classified as an alicyclic group, aromatic group, or heterocyclic group. The term “alicyclic group” means a cyclic hydrocarbon group having properties resembling those of aliphatic groups. The term “aromatic group” or “aryl group” means a mono- or polycyclic aromatic hydrocarbon group, and may include one or more heteroatoms, and which are further defined below. The term “heterocyclic group” means a closed ring hydrocarbon in which one or more of the atoms in the ring are an element other than carbon (e.g., nitrogen, oxygen, sulfur, etc.), and are further defined below.

“Organic groups” may be functionalized or otherwise comprise additional functionalities associated with the organic group, such as carboxyl, amino, hydroxyl, and the like, which may be protected or unprotected. For example, the phrase “alkyl group” is intended to include not only pure open chain saturated hydrocarbon alkyl substituents, such as methyl, ethyl, propyl, t-butyl, and the like, but also alkyl substituents bearing further substituents known in the art, such as hydroxy, alkoxy, alkylsulfonyl, halogen atoms, cyano, nitro, amino, carboxyl, etc. Thus, “allyl group” includes ethers, esters, haloalkyls, nitroalkyls, carboxyalkyls, hydroxyalkyls, sulfoalkyls, etc.

The terms “halo” and “halogen” refer to the fluoro, chloro, bromo or iodo groups. There can be one or more halogen, which are the same or different. Halogens of particular interest include chloro and bromo groups.

The term “haloalkyl” refers to an alkyl group as defined above that is substituted by one or more halogen atoms. The halogen atoms may be the same or different. The term “dihaloalkyl” refers to an alkyl group as described above that is substituted by two halo groups, which may be the same or different. The term “trihaloalkyl” refers to an allyl group as describe above that is substituted by three halo groups, which may be the same or different. The term “perhaloalkyl” refers to a haloalkyl group as defined above wherein each hydrogen atom in the alkyl group has been replaced by a halogen atom. The term “perfluoroalkyl” refers to a haloalkyl group as defined above wherein each hydrogen atom in the allyl group has been replaced by a fluoro group.

The term “cycloalkyl” means a mono-, bi-, or tricyclic saturated ring that is fully saturated or partially unsaturated. Examples of such a group included cyclopropyl, cyclobutyl, cyclopentyl, cyclohexyl, cycloheptyl, adamantyl, cyclooctyl, cis- or trans decalin, bicyclo [2.2.1]hept-2-ene, cyclohex-1-enyl, cyclopent-1-enyl, 1,4-cyclooctadienyl, and the like.

The term “(cycloalkyl)alkyl” means the above-defined alkyl group substituted for one of the above cycloalkyl rings. Examples of such a group include (cyclohexyl)methyl, 3-(cyclopropyl)-n-propyl, 5-(cyclopentyl)hexyl, 6-(adamantyl)hexyl, and the like.

The term “substituted phenyl” specifies a phenyl group substituted with one or more moieties, and in some instances one, two, or three moieties, chosen from the groups consisting of halogen, hydroxy, protected hydroxy, cyano, nitro, trifluoromethyl, C₁ to C₇ allyl, C₁ to C₇ alkoxy, C₁ to C₇ acyl, C₁ to C₇ acyloxy, carboxy, oxycarboxy, protected carboxy, carboxymethyl, protected carboxymethyl, hydroxymethyl, protected hydroxymethyl, amino, protected amino, (monosubstituted)amino, protected (monosubstituted)amino, (disubstituted)amino, carboxamide, protected carboxamide, N—(C₁ to C₆ alkyl)carboxamide, protected N—(C₁ to C₆ allyl)carboxamide, N,N-di(C₁ to C₆ alkyl)carboxamide, trifluoromethyl, N—((C₁ to C₆ allyl)sulfonyl)amino, N-(phenylsulfonyl)amino or phenyl, substituted or unsubstituted, such that, for example, a biphenyl or naphthyl group results.

Examples of the term “substituted phenyl” includes a mono- or di(halo)phenyl group such as 2, 3 or 4-chlorophenyl, 2,6-dichlorophenyl, 2,5-dichlorophenyl, 3,4-dichlorophenyl, 2, 3 or 4-bromophenyl, 3,4-dibromophenyl, 3-chloro-4-fluorophenyl, 2, 3 or 4-fluorophenyl and the like; a mono or di(hydroxy)phenyl group such as 2, 3, or 4-hydroxyphenyl, 2,4-dihydroxyphenyl, the protected-hydroxy derivatives thereof and the like; a nitrophenyl group such as 2, 3, or 4-nitrophenyl; a cyanophenyl group, for example, 2, 3 or 4-cyanophenyl; a mono- or di(alkyl)phenyl group such as 2, 3, or 4-methylphenyl, 2,4-dimethylphenyl, 2, 3 or 4-(iso-propyl)phenyl, 2, 3, or 4-ethylphenyl, 2, 3 or 4-(n-propyl)phenyl and the like; a mono or di(alkoxy)phenyl group, for example, 2,6-dimethoxyphenyl, 2, 3 or 4-(isopropoxy)phenyl, 2, 3 or 4-(t-butoxy)phenyl, 3-ethoxy-4-methoxyphenyl and the like; 2, 3 or 4-trifluoromethylphenyl; a mono- or dicarboxyphenyl or (protected carboxy)phenyl group such as 2, 3 or 4-carboxyphenyl or 2,4-di(protected carboxy)phenyl; a mono- or di(hydroxymethyl)phenyl or (protected hydroxymethyl)phenyl such as 2, 3 or 4-(protected hydroxymethyl)phenyl or 3,4-di(hydroxymethyl)phenyl; a mono- or di(aminomethyl)phenyl or (protected aminomethyl)phenyl such as 2, 3 or 4-(aminomethyl)phenyl or 2,4-(protected aminomethyl)phenyl; or a mono- or di(N-(methylsulfonylamino))phenyl such as 2, 3 or 4-(N-(methylsulfonylamino))phenyl. Also, the term “substituted phenyl” represents disubstituted phenyl groups wherein the substituents are different, for example, 3-methyl-4-hydroxyphenyl, 3-chloro-4-hydroxyphenyl, 2-methoxy-4-bromophenyl, 4-ethyl-2-hydroxyphenyl, 3-hydroxy-4-nitrophenyl, 2-hydroxy-4-chlorophenyl and the like.

The term “(substituted phenyl)alkyl” means one of the above substituted phenyl groups attached to one of the above-described alkyl groups. Examples of include such groups as 2-phenyl-1-chloroethyl, 2-(4′-methoxyphenyl)ethyl, 4-(2′,6′-dihydroxy phenyl)n-hexyl, 2-(5′-cyano-3′-methoxyphenyl)n-pentyl, 3-(2′,6′-dimethylphenyl)n-propyl, 4-chloro-3-aminobenzyl, 6-(4′-methoxyphenyl)-3-carboxy(n-hexyl), 5-(4′-aminomethylphenyl)-3-(aminomethyl)n-pentyl, 5-phenyl-3-oxo-n-pent-1-yl, (4-hydroxynapth-2-yl)methyl and the like.

As noted above, the term “aromatic” or “aryl” refers to six membered carbocyclic rings. Also as noted above, the term “heteroaryl” denotes optionally substituted five-membered or six-membered rings that have 1 to 4 heteroatoms, such as oxygen, sulfur and/or nitrogen atoms, in particular nitrogen, either alone or in conjunction with sulfur or oxygen ring atoms.

Furthermore, the above optionally substituted five-membered or six-membered rings can optionally be fused to an aromatic 5-membered or 6-membered ring system. For example, the rings can be optionally fused to an aromatic 5-membered or 6-membered ring system such as a pyridine or a triazole system, and preferably to a benzene ring.

The following ring systems are examples of the heterocyclic (whether substituted or unsubstituted) radicals denoted by the term “heteroaryl”: thienyl, furyl, pyrrolyl, pyrrolidinyl, imidazolyl, isoxazolyl, triazolyl, thiadiazolyl, oxadiazolyl, tetrazolyl, thiatriazolyl, oxatriazolyl, pyridyl, pyrimidyl, pyrazinyl, pyridazinyl, oxazinyl, triazinyl, thiadiazinyl tetrazolo, 1,5-[b]pyridazinyl and purinyl, as well as benzo-fused derivatives, for example, benzoxazolyl, benzthiazolyl, benzimidazolyl and indolyl.

Substituents for the above optionally substituted heteroaryl rings are from one to three halo, trihalomethyl, amino, protected amino, amino salts, mono-substituted amino, di-substituted amino, carboxy, protected carboxy, carboxylate salts, hydroxy, protected hydroxy, salts of a hydroxy group, lower alkoxy, lower alkylthio, alkyl, substituted alkyl, cycloalkyl, substituted cycloallyl, (cycloallyl)alkyl, substituted (cycloalkyl)alkyl, phenyl, substituted phenyl, phenylallyl, and (substituted phenyl)alkyl. Substituents for the heteroaryl group are as heretofore defined, or in the case of trihalomethyl, can be trifluoromethyl, trichloromethyl, tribromomethyl, or triiodomethyl. As used in conjunction with the above substituents for heteroaryl rings, “lower alkoxy” means a C₁ to C₄ alkoxy group, similarly, “lower alkylthio” means a C₁ to C₄ alkylthio group.

The term “(monosubstituted)amino” refers to an amino group with one substituent chosen from the group consisting of phenyl, substituted phenyl, alkyl, substituted alkyl, C₁ to C₄ acyl, C₂ to C₇ alkenyl, C₂ to C₇ substituted alkenyl, C₂ to C₇ alkynyl, C₇ to C₁₆ alkylaryl, C₇ to C₁₆ substituted alkylaryl and heteroaryl group. The (monosubstituted) amino can additionally have an amino-protecting group as encompassed by the term “protected (monosubstituted)amino.” The term “(disubstituted)amino” refers to amino groups with two substituents chosen from the group consisting of phenyl, substituted phenyl, allyl, substituted allyl, C₁ to C₇ acyl, C₂ to C₇ alkenyl, C₂ to C₇ alkynyl, C₇ to C₁₆ alkylaryl, C₇ to C₁₆ substituted alkylaryl and heteroaryl. The two substituents can be the same or different.

The term “heteroaryl(allyl)” denotes an allyl group as defined above, substituted at any position by a heteroaryl group, as above defined.

“Optional” or “optionally” means that the subsequently described event, circumstance, feature or element may, but need not, occur, and that the description includes instances where the event or circumstance occurs and instances in which it does not. For example, “heterocyclo group optionally mono- or di-substituted with an alkyl group” means that the alkyl may, but need not, be present, and the description includes situations where the heterocyclo group is mono- or disubstituted with an alkyl group and situations where the heterocyclo group is not substituted with the alkyl group.

Compounds that have the same molecular formula but differ in the nature or sequence of bonding of their atoms or the arrangement of their atoms in space are termed “isomers.” Isomers that differ in the arrangement of their atoms in space are termed “stereoisomers.” Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture.”

The compounds described herein may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see, e.g., the discussion in Chapter 4 of “Advanced Organic Chemistry”, 4th edition J. March, John Wiley and Sons, New York, 1992).

### Overview

The invention provides methods and composition for modulating angiogenesis and/or cell migration through modulation of an aquaporin, particularly aquaporin-1. The invention is based in part on the surprising discovery that disruption of AQP1 function reduces microvessel proliferation in tumors, and also inhibits cell migration. Since defects in AQP1 are not generally associated with serious clinical consequences, as described above, AQP1 provides an excellent target for agents for inhibition of cellular proliferation, e.g., anti-cancer agents.

As described in the examples in more detail, the invention is based on the discovery that AQP1 null mice are remarkably impaired in tumor growth after subcutaneous or intracranial tumor cell implantation, with reduced tumor vascularity and extensive necrosis. Further, and without wishing to be bound to theory, a novel mechanism for the impaired angiogenesis was established from cell culture studies. Although adhesion and proliferation were similar in primary cultures of aortic endothelia from wildtype versus AQP1 null mice, cell migration was greatly impaired in AQP1 deficient cells, with abnormal vessel formation in vitro. Stable transfection of non-endothelial cells with AQP1, or a structurally different water-selective transporter (AQP4), accelerated cell migration and in vitro wound healing. Motile AQP1-expressing cells exhibited prominent membrane ruffles at the leading edge with polarization of AQP1 protein to lamellipodia, where rapid water fluxes occur. These observations support a fundamental role of water channels in cell migration, which is central to diverse biological phenomena including angiogenesis, wound healing, tumor spread and organ regeneration.

In one aspect the invention provides methods of screening compounds for identifying agents that modulate AQP1 activity. Agents that inhibit AQP1 activity can be useful as inhibitors of cellular proliferation and migration (e.g., as anti-cancer agents). Agents that enhance AQP1 activity can be useful to promote cellular proliferation and migration (e.g., as in wound healing and organ regeneration). In another aspect the invention provides methods for modulating angiogenesis and/or cell migration in a subject.

The invention will now be described in more detail.

### Compositions

In certain embodiments, the AQP1 inhibiting compound of the present invention is a pyrazole containing compounds described herein, which comprises a substituted pyrazole group. In specific embodiments, the subject compound are generally described by Formula (I) as follows:

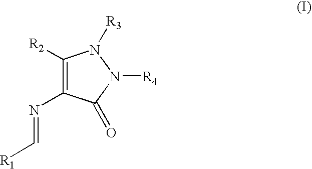

wherein R₁ is independently selected from a substituted or unsubstituted phenyl group; R₂ is independently chosen from a hydrogen, or an alkyl group such as a substituted or unsubstituted, saturated linear or branched hydrocarbon group or chain (e.g., C₁ to C₈) including, e.g., methyl, ethyl, isopropyl, tert-butyl, heptyl, n-octyl, dodecyl, octadecyl, amyl, 2-ethylhexyl; R₃ is independently chosen from a hydrogen, or an alkyl group such as a substituted or unsubstituted, saturated linear or branched hydrocarbon group or chain (e.g., C₁ to C₈) including, e.g., methyl, ethyl, isopropyl, tert-butyl, heptyl, n-octyl, dodecyl, octadecyl, amyl, 2-ethylhexyl; and R₄ is independently selected from a substituted or unsubstituted phenyl group; or a pharmaceutically acceptable derivative thereof, as an individual stereoisomer or a mixture thereof. Exemplary substitutions for R₁, R₂, R₃, and R₄ are described in more detail below.

In representative embodiments, R₁ is independently chosen from an unsubstituted phenyl group, a unsubstituted biphenyl group, or a substituted phenyl group, such as a mono-, di- or tri-(alkyl)phenyl group, mono-, di- or tri-(alkoxy)phenyl group, a mono-, di- or tri-(hydroxy)phenyl group, a mono-, di- or tri-(halo)phenyl group, a mono-, di- or tri-(alkenyl)phenyl group, a mono-, di- or tri-(nitro)phenyl group, a mono(alkyl)-mono(alkoxy)phenyl group, a mono a di(alkoxy)-mono(halo)phenyl group, a mono(nitro)-mono(halo)-mono(hydroxy)phenyl group, such as a 2-(nitro)-4-(bromo)-5-(hydroxy)phenyl group; R₂ is independently chosen from a hydrogen or an lower alkyl group (e.g., C₁-C₄), such as a methyl group; R₃ is independently a hydrogen or a lower alkyl group (e.g., C₁-C₄), such as a methyl group; and R₄ is an unsubstituted phenyl group.

In certain embodiments, the AQP1 inhibiting compound of the present invention is a substituted piperazine containing compounds described herein. In specific embodiments, the subject compound are generally described by Formula (II) as follows:

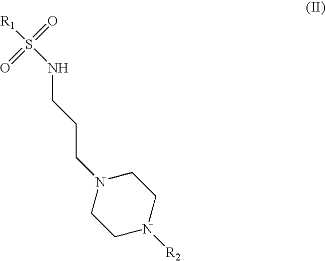

wherein R₁ is independently selected from a substituted or unsubstituted phenyl group, a substituted or unsubstituted heteroaromatic group, such as a substituted or unsubstituted quinolinyl group, a substituted or unsubstituted anthracenyl group, and a substituted or unsubstituted naphthalenyl group; and R₂ is independently selected from a substituted or unsubstituted phenyl group; or a pharmaceutically acceptable derivative thereof, as an individual stereoisomer or a mixture thereof. Exemplary substitutions for R₁ and R₂ are described in more detail below.

In representative embodiments, R₁ is independently selected from a substituted or unsubstituted heteroaromatic group, such as a substituted or unsubstituted quinolinyl group, a substituted or unsubstituted anthracenyl group, and a substituted or unsubstituted naphthalenyl group; and R₂ is independently chosen from an unsubstituted phenyl group, a unsubstituted biphenyl group, or a substituted phenyl group, such as a mono-, di- or tri-(alkyl)phenyl group, mono-, di- or tri-(alkoxy)phenyl group, a mono-, di- or tri-(hydroxy)phenyl group, a mono-, di- or tri-(halo)phenyl group such as a 2-(fluoro)phenyl group, a mono-, di- or tri-(alkenyl)phenyl group, a mono-, di- or tri-(nitro)phenyl group, a mono(alkyl)-mono(alkoxy)phenyl group, a mono a di(alkoxy)-mono(halo)phenyl group, a mono(nitro)-mono(halo)-mono(hydroxy)phenyl group.

In some embodiments of the invention, the compounds may comprise a formula of the following:

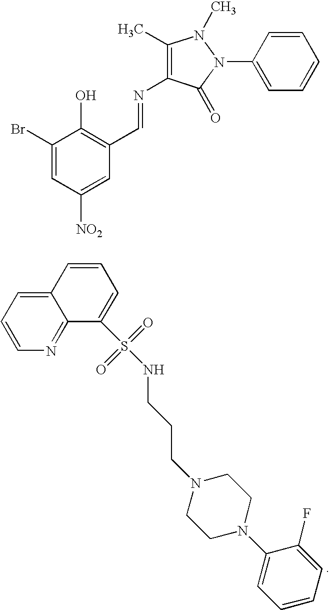

Pharmaceutical Preparations Containing Compounds of the Invention

Also provided by the invention are pharmaceutical preparations of the subject compounds described above. The subject compounds can be incorporated into a variety of formulations for therapeutic administration by a variety of routes. More particularly, the compounds of the present invention can be formulated into pharmaceutical compositions by combination with appropriate, pharmaceutically acceptable carriers, diluents, excipients and/or adjuvants, and may be formulated into preparations in solid, semi-solid, liquid or gaseous forms, such as tablets, capsules, powders, granules, ointments, solutions, suppositories, injections, inhalants and aerosols. In most embodiments, the formulations are free of detectable DMSO (dimethyl sulfoxide), which is not a pharmaceutically acceptable carrier, diluent, excipient, or adjuvant, particularly in the context of routes of administration other than transdermal routes. Where the formulation is for transdermal administration, the compounds are preferably formulated either without detectable DMSO or with a carrier in addition to DMSO. The formulations may be designed for administration to subjects or patients in need thereof via a number of different routes, including oral, buccal, rectal, parenteral, intraperitoneal, intradermal, intratracheal, etc., administration.

Pharmaceutically acceptable excipients usable with the invention, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents. Actual methods of preparing such dosage forms are known, or will be apparent, to those skilled in the art. See, e.g., Remington's Pharmaceutical Sciences, Mack Publishing Company, Easton, Pa., 17th edition, 1985; Remington: The Science and Practice of Pharmacy, A. R. Gennaro, (2000) Lippincott, Williams & Willins. The composition or formulation to be administered will, in any event, contain a quantity of the agent adequate to achieve the desired state in the subject being treated.

Dosage Forms of Compounds of the Invention

In pharmaceutical dosage forms, the subject compounds of the invention may be administered in the form of their pharmaceutically acceptable salts, or they may also be used alone or in appropriate association, as well as in combination, with other pharmaceutically active compounds. The following methods and excipients are merely exemplary and are in no way limiting.

The agent can be administered to a host using any available conventional methods and routes suitable for delivery of conventional drugs, including systemic or localized routes. In general, routes of administration contemplated by the invention include, but are not necessarily limited to, enteral, parenteral, or inhalational routes, such as intrapulmonary or intranasal delivery.

Conventional and pharmaceutically acceptable routes of administration include intranasal, intrapulmonary intramuscular, intratracheal, intratumoral, subcutaneous, intradermal, topical application, intravenous, rectal, nasal, oral and other parenteral routes of administration. Routes of administration may be combined, if desired, or adjusted depending upon the agent and/or the desired effect. The composition can be administered in a single dose or in multiple doses.

For oral preparations, the subject compounds can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

Parenteral routes of administration other than inhalation administration include, but are not necessarily limited to, topical, transdermal, subcutaneous, intramuscular, intraorbital, intracapsular, intraspinal, intrasternal, and intravenous routes, i.e., any route of administration other than through the alimentary canal. Parenteral administration can be carried to effect systemic or local delivery of the agent. Where systemic delivery is desired, administration typically involves invasive or systemically absorbed topical or mucosal administration of pharmaceutical preparations.

Methods of administration of the agent through the skin or mucosa include, but are not necessarily limited to, topical application of a suitable pharmaceutical preparation, transdermal transmission, injection and epidermal administration. For transdermal transmission, absorption promoters or iontophoresis are suitable methods. Iontophoretic transmission may be accomplished using commercially available “patches” which deliver their product continuously via electric pulses through unbroken skin for periods of several days or more.

The subject compounds of the invention can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The agent can also be delivered to the subject by enteral administration. Enteral routes of administration include, but are not necessarily limited to, oral and rectal (e.g., using a suppository) delivery.

Furthermore, the subject compounds can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Dosages of the Compounds of the Invention

Depending on the subject and condition being treated and on the administration route, the subject compounds may be administered in dosages of, for example, 0.1 μg to 10 mg/kg body weight per day. The range is broad, since in general the efficacy of a therapeutic effect for different mammals varies widely with doses typically being 20, 30 or even 40 times smaller (per unit body weight) in man than in the rat. Similarly the mode of administration can have a large effect on dosage. Thus, for example, oral dosages may be about ten times the injection dose. Higher doses may be used for localized routes of delivery.

A typical dosage may be a solution suitable for intravenous administration; a tablet taken from two to six times daily, or one time-release capsule or tablet taken once a day and containing a proportionally higher content of active ingredient, etc. The time-release effect may be obtained by capsule materials that dissolve at different pH values, by capsules that release slowly by osmotic pressure, or by any other known means of controlled release.

Those of skill in the art will readily appreciate that dose levels can vary as a function of the specific compound, the severity of the symptoms and the susceptibility of the subject to side effects. Preferred dosages for a given compound are readily determinable by those of skill in the art by a variety of means.

Although the dosage used will vary depending on the clinical goals to be achieved, a suitable dosage range is one which provides up to about 1 μg to about 1,000 μg or about 10,000 μg of subject composition to reduce a symptom in a subject animal.

Unit dosage forms for oral or rectal administration such as syrups, elixirs, and suspensions tablet or suppository, contains a predetermined amount of the composition containing one or more compounds of the invention. Similarly, unit dosage forms for injection or intravenous administration may comprise the compound (s) in a composition as a solution in sterile water, may be provided wherein each dosage unit, for example, teaspoonful, tablespoonful, normal saline or another pharmaceutically acceptable carrier.

Combination Therapy Using the Compounds of the Invention

For use in the subject methods, the subject compounds may be formulated with or otherwise administered in combination with other pharmaceutically active agents, including other agents that modulate angiogenesis and/or cell migration, preferably through a mechanism of action that does not interfere with an aquaporin-modulating compound. Aquaporin-modulating compounds may be used to provide an increase in the effectiveness of another chemical, such as a pharmaceutical (e.g., other angiogenesis-modulating or cell migration-modulating agents), or a decrease in the effectiveness of another chemical, such as a pharmaceutical (e.g., where inhibition of angiogenesis is desired, to decrease effectiveness of an angiogenic agent), that is necessary to produce the desired biological effect.

Examples of other agents for use in combination therapy include, but are not limited to, thalidomide, marimastat, COL-3, BMS-275291, squalamine, 2-ME, SU6668, neovastat, Medi-522, EMD121974, CAI, celecoxib, interleukin-12, IM862, TNP470, avastin, gleevac, herceptin, interferon beta, and mixtures thereof. Examples of chemotherapeutic agents for use in combination therapy include, but are not limited to, daunorubicin, daunomycin, dactinomycin, doxorubicin, epirubicin, idarubicin, esorubicin, bleomycin, mafosfamide, ifosfamide, cytosine arabinoside, bis-chloroethylnitrosurea, busulfan, mitomycin C, actinomycin D, mithramycin, prednisone, hydroxyprogesterone, testosterone; tamoxifen, dacarbazine, procarbazine, hexamethylmelamine, pentamethylmelamine, mitoxantrone, amsacrine, chlorambucil, methylcyclohexylnitrosurea, nitrogen mustards, melphalan, cyclophosphamide, 6-mercaptopurine, 6-thioguanine, cytarabine, 5-azacytidine, hydroxyurea, deoxycoformycin, 4-hydroxyperoxycyclophosphor-amide, 5-fluorouracil (5-FU), 5-fluorodeoxyuridine (5-FUdR), methotrexate (MTX), colchicine, taxol, vincristine, vinblastine, etoposide (VP-16), trimetrexate, irinotecan, topotecan, gemcitabine, teniposide, cisplatin and diethylstilbestrol (DES).

In the context of a combination therapy, aquaporin-modulating compounds may be administered by the same route of administration (e.g. intrapulmonary, oral, enteral, etc.) that the compounds are administered. In the alternative, the compounds for use in combination therapy with aquaporin-modulating compounds may be administered by a different route of administration that the compounds are administered.

Kits

Kits with unit doses of the subject compounds, usually in oral or injectable doses, are provided. In such kits, in addition to the containers containing the unit doses will be an informational package insert describing the use and attendant benefits of the drugs in treating pathological condition of interest. Preferred compounds and unit doses are those described herein above.

### Methods

Methods for Screening an Agent for Activity in Modulating an Aquaporin-Mediated Activity

The invention provides methods screening an agent for activity in modulating an aquaporin-mediated activity. In general, the method involves contacting the cell with a compound in an amount effective to aquaporin-mediated activity, such as angiogenesis or cell migration. In general, the method involves contacting a cell with a compound in an amount effective to modulate the aquaporin-mediated activity of the cell and determining whether the compound affects at least one cell function associated with angiogenesis, such as cell proliferation, cell adhesion, and cell migration. In some embodiments, the modulation of an aquaporin-mediated activity results in a decrease in angiogenesis. In other embodiments, the modulation of an aquaporin-mediated activity results in an increase in angiogenesis. In still other embodiments, the modulation of an aquaporin-mediated activity results in a decrease in cell migration. In still other embodiments, the modulation of an aquaporin-mediated activity results in an increase in cell migration.

In one embodiment, the subject method is carried out by culturing a mammalian cell expressing an aquaporin, particularly aquaporin-1 in the presence of a candidate agent, and determining the level of at least one of cell proliferation, cell adhesion, and cell migration, wherein an increase or decrease in cell proliferation, cell adhesion or cell migration as compared to the absence of the candidate agent indicates the candidate agent modulates the aquaporin-mediated activity.

Determining the level of cell proliferation, cell adhesion, and cell migration can be carried out by any number of methods well known in the art, such as those described in greater detail in the examples section below.

In another embodiment, the subject method can be carried out by measuring water permeability of a mammalian cell expressing an aquaporin, such as AQP1, and a fluorescent protein, such as green-fluorescent protein, in the presence of a candidate agent as compared to the absence of the candidate agent indicates the candidate agents modulates the aquaporin-mediated activity. Water transport may be assayed from the kinetics of cell swelling in response to dilution of the extracellular medium with water (typically 1:1). The cell swelling may also be following from the time course of fluorescence in a plate reader. An exemplary method of measuring water permeability in living cells and complex tissues is provided in Verkman et al., J. Membrane Biol. 173:73-87 (2000).

In yet another embodiment, the subject method is carried out by an in vivo tumor-independent model that involves subcutaneous implantation of Matrigel pellet containing an angiogenic compound (e.g., bFGF) and a candidate agent. In such embodiments, the Matrigel pellet containing the angiogeneic compound and the candidate gent are subcutaneously implanted in a suitable animal host. Following a period of time sufficient to allow the occurrence of angiogenesis, such as about 3 days, about 4 days, about 5 days, and the like, the Matrigel pellet is removed from the animal host and examined for characteristics associated with angiogenesis (e.g., vessel formation) as compared to a control (i.e., a Matrigel pellet containing the angiogeneic compound but lacking the candidate agent). Such characteristics include, but are not limited to, formation of vessel-like structures, total Matrigen hemoglobin levels, and the like. Animal hosts suitable for use in such embodiments include, but are not limited to, mice, rats, guinea pigs, and the like. Angiogeneic compounds suitable for use in such embodiments include, but are not limited to, platelet-derived growth factor (PDGF) fibroblast growth factor (bFGF), vascular endothelial growth factor (VEGF), and the like.

In yet another embodiment, in silico modeling can be used to screen 3-dimensional libraries of compounds for activity in binding to and modulating the activity of an aquaporin, particularly AQP1. An exemplary in silico modeling program suitable for use with the subject method is the PREDICT™ 3D Modeling Technology (Predix Pharmaceuticals, Woburn Mass.), described in greater detail in Becker et al., PNAS 101(31):11304-11309 (2004). In some embodiments, the candidate agent will bind to AQP1 and disrupt the activity of AQP1, i.e., the compound will decrease the activity of AQP1. In other embodiments, the candidate agent will bind to AQP1 and increase the activity of AQP1.

In many embodiments, the AQP1 is present on the plasma membrane of the cell. Methods of detecting AQP1 presence on the plasma membrane are well lnown in the art and can include but are not limited to, for example, labeling a molecule that binds to AQP1 with a fluorescent, chemical or biological tag. Examples of molecules that bind to AQP1 include, without limitation, antibodies (monoclonal and polyclonal), FAB fragments, humanized antibodies and chimeric antibodies.

In most embodiments, the aquaporin mediated-activity is modulated (increased or decreased) by up to about 10%, by up to about 20%, by up to about 50%, by up to about 100%, by up to about 150%, by up to about 200%, by up to about 300%, by up to about 400%, by up to about 500%, by up to about 800%, or up to about 1000% or more.

Suitable cells include those cells that have an endogenous or introduced AQP1 gene. Suitable cells include mammalian cell systems (e.g., COS, CHO, BHK, 293, 3T3 cells etc.) containing constructs that have an expression cassette for expression of AQP1. The cell used in the subject methods may be a cell present in vivo, ex vivo, or in vitro. As used herein, the term “expression cassette” is meant to denote a genetic sequence, e.g. DNA or RNA, that codes for an aquaporin, e.g., AQP1. Methods of introducing an expression cassette into a cell are well known in the art, see for example, Sambrook et al., Molecular Cloning: A Laboratory Manual. Cold Spring Harbor Laboratory Press, NY, Vol. 1, 2, 3 (1989).

Screening to determine drugs that do not significantly modulate angiogenesis and/or cell migration is also of interest. Assays of the invention make it possible to identify agents (such as a gene product or a compound) which ultimately: (1) have a positive effect with respect to modulating angiogenesis and/or cell migration and as such are potential therapeutics, e.g. agents which are suitable for use in treating a cellular proliferative disease; or (2) have an adverse affect with respect to the angiogenesis and/or cell migration and as such should be avoided as therapeutic agents (e.g., to screen candidate agents for toxicity to mammalian cells).

A variety of different candidate agents may be screened by the above methods. Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For a example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Moreover, screening may be directed to known pharmacologically active compounds and chemical analogs thereof, or to new agents with unknown properties such as those created through rational drug design.

The above screening methods may be part of a multi-step screening process of evaluating candidate agents for their efficacy (and safety) in the treatment of cellular proliferative diseases, e.g., cancer, in mammalian hosts, e.g. humans. In multi-step screening processes of the subject invention, a candidate compound or library of compounds is subjected to screening in a second in vivo model, e.g. a mouse model, following screening in the subject cell lines. Following the initial screening in the cell lines of the subject invention, the positive compounds are then screened in non-human mammalian animal models. In addition, a pre in vivo screening step may be employed, in which the compound is first subjected to an in vitro screening assay for its potential as a therapeutic agent in the treatment of cellular proliferative disease. Any convenient in vitro screening assay may be employed, where a variety of suitable in vitro screening assays are known to those of skill in the art.

Methods of Modulating Angiogenesis and/or Cell Migration

The invention also provides methods of modulating cellular proliferation and/or migration, e.g., modulation of angiogenesis.

In one embodiment, the invention provides methods for inhibiting angiogenesis and/or cell migration in a subject having a condition associated with cellular proliferative disease, e.g., cancer. In general, the method involves administering to the subject a compound of the invention in an amount effective to modulate an aquaporin-mediated activity and thereby treat the condition. In an embodiment of particular interest, a compound of the invention is administered in combination with a second AQP1 modulator, e.g., a compound that decreases AQP1 activity.

Agents that act to inhibit AQP1 activity are useful in the treatment of a cellular proliferative disease, e.g., any condition, disorder or disease, or symptom of such condition, disorder, or disease that results from the uncontrolled proliferation of cells, e.g., cancer. Cancer is an example of a condition that is treatable using the compounds of the invention. Use of the compounds of the invention in combination with a second compound for use in treatment of a cellular proliferative disease is of particular interest. Exemplary cancers suitable for treatment with the subject methods include colorectal cancer, non-small cell lung cancer, small cell lung cancer, ovarian cancer, breast cancer, head and neck cancer, renal cell carcinoma, and the like.

Subjects suitable for treatment with a method of the present invention involving inhibition of AQP1 activity include individuals having a cellular proliferative disease, such as a neoplastic disease (e.g., cancer). Cellular proliferative disease is characterized by the undesired propagation of cells, including, but not limited to, neoplastic disease conditions, e.g., cancer. Examples of cellular proliferative disease include, but not limited to, abnormal stimulation of endothelial cells (e.g., atherosclerosis), solid tumors and tumor metastasis, benign tumors, for example, hemangiomas, acoustic neuromas, neurofibromas, trachomas, and pyogenic granulomas, vascular malfunctions, abnormal wound healing, inflammatory and immune disorders, Bechet's disease, gout or gouty arthritis, abnormal angiogenesis accompanying, for example, rheumatoid arthritis, psoriasis, diabetic retinopathy, other ocular angiogenic diseases such as retinopathy of prematurity (retrolental fibroplastic), macular degeneration, corneal graft rejection, neurovascular glaucoma and Oster Webber syndrome, psoriasis, restinosis, fungal, parasitic and viral infections such cytomegaloviral infections. Subjects to be treated according to the methods of the invention include any individual having any of the above-mentioned disorders.

Agents that act to promote AQP1 activity are useful in treatment of conditions in which stimulation of cellular proliferation is desirable. For example, the AQP1 enhancing agents can be used to treat a variety of conditions that would benefit from stimulation of angiogenesis, stimulation of vasculogenesis, increased blood flow, and/or increased vascularity, organ regeneration, and wound healing.

Examples of conditions and diseases amenable to treatment according to this method of the invention include any condition associated with an obstruction of a blood vessel, e.g., obstruction of an artery, vein, or of a capillary system. Specific examples of such conditions or disease include, but are not necessarily limited to, coronary occlusive disease, carotid occlusive disease, arterial occlusive disease, peripheral arterial disease, atherosclerosis, myointimal hyperplasia (e.g., due to vascular surgery or balloon angioplasty or vascular stenting), thromboangiitis obliterans, thrombotic disorders, vasculitis, and the like. Examples of conditions or diseases that can be prevented using the methods of the invention include, but are not necessarily limited to, heart attack (myocardial infarction) or other vascular death, stroke, death or loss of limbs associated with decreased blood flow, and the like.

Other forms of therapeutic angiogenesis include, but are not necessarily limited to, the use of nicotine receptor agonists to accelerate healing of wounds or ulcers; to improve the vascularization of skin grafts or reattached limbs so as to preserve their function and viability; to improve the healing of surgical anastomoses (e.g., as in re-connecting portions of the bowel after gastrointestinal surgery); and to improve the growth of skin or hair.

In addition, agents that act to promote AQP1 activity are useful in treatment of conditions in which stimulation of the water channel properties of AQP1. For example, the AQP1 enhancing agents can be used to treat a variety of conditions that would benefit from stimulation of AQP1 such as a diuretic in congestive heart failure and as a antihypertensive, and in treatment of glaucoma.

The invention should not be construed to be limited solely to the treatment of patients having a cellular proliferative disease. Rather, the invention should be construed to include the treatment of patients having conditions or disease associates with aberrant angiogenesis and/or cell migration with similar characteristics.

Subjects suitable for treatment using the methods of the invention include any animal with a condition amenable to treatment by modulation of AQP1 activity including cellular proliferative disease condition (by administration of an AQP1 inhibitor) and Exemplary subjects include mammals, e.g., non-human primates (e.g., monkey, chimpanzee, gorilla, and the like), rodents (e.g., rats, mice, gerbils, hamsters, ferrets, and the like), lagomorphs, swine (e.g., pig, miniature pig), equine, canine, feline, and the like. Large animals are of particular interest. Transgenic mammals may also be used, e.g. mammals that have a chimeric gene sequence. Methods of making transgenic animals are well known in the art, see, for example, U.S. Pat. No. 5,614,396.

Such subjects may be tested in order to assay the activity and efficacy of the subject compounds. Significant improvements in one or more of parameters is indicative of efficacy. It is well within the skill of the ordinary healthcare worker (e.g., clinician) provide adjust dosage regimen and dose amounts to provide for optimal benefit to the patient according to a variety of factors (e.g., patient-dependent factors such as the severity of the disease and the like), the compound administered, and the like).

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

The following methods and materials are used in the examples below.

Methods

Mice. AQP1 (AQP1⁻/AQP1⁻) and AQP3 (AQP3⁻/AQP3⁻) null mice were generated by targeted gene disruption as described previously (Ma et al. Proc Natl Acad Sci USA 97, 4386-91 (2000); Ma, et al. J Biol Chem 273, 4296-9 (1998)). Experiments were performed on weight and sex matched wildtype and AQP1 null mice in CD1, C57/BL6, or BALB/C genetic backgrounds. Investigators were blinded to the genotype for all experiments. Protocols were approved by the University of California Committee on Animal Research.

Tumor cell implantation. B16F10 melanoma cells (ATCC) were cultured in DMEM supplemented with 4 mM L-glutamine, 4.5 g/L dextrose and 10% fetal bovine serum. 10⁶ B16F10 cells suspended in 200 μL PBS were injected subcutaneously into anesthetized mice between the shoulder blades. Tumor length (L) and width (W) were measured using a caliper and tumor volume was estimated (Egami et al. J Clin Invest 112, 67-75 (2003)) as (W²×L). In some mice, 5 μL PBS containing 10⁵ B16F10 cells were stereotactically injected into the right cerebral hemisphere as previously described (Papadopoulos et al. Faseb J 18, 1291-3 (2004)). Brain tumor volume was measured 7 days post-implantation by summing the tumor areas of 1 mm thick coronal brain slices.

In vivo Matrigel assay. Each anesthetized mouse received two 0.5 mL injections of Matrigel (without supplement vs. supplemented with 20 nM bFGF plus 25 mg heparin, BD Biosciences) under abdominal skin (Yao et al. Blood 93, 1612-21 (1999).). Matrigel pellets were harvested after 5 d, digested with dispase (Sigma-Aldrich, St. Louis, Mo.) and hemoglobin content was determined by Drabkin's method (Ricca Chemical Company) according to the manufacturer's instructions. Matrigel pellets from two wildtype and two AQP1 null mice were examined histologically after hematoxylin-eosin staining.

Histology. Cells, tumors and lungs were fixed in formalin or 4% paraformaldehyde, and embedded in paraffin. Sections were immunostained using a polyclonal rabbit anti-AQP1 antibody (Ma, et al. J Biol Chem 273, 4296-9 (1998)) followed by biotinylated goat anti-rabbit antibody or biotinylated isolectin-B4 (Vector Labs). Isolectin-B4 selectively stains mouse blood vessels (Kawamoto et al. Circulation 107, 461-8 (2003)). Sections were then treated with avidin-horseradish peroxidase and diaminobenzidine, incubated with 3% H₂O₂ to bleach melanin, and counterstained with hematoxylin. Certain AQP1 immunohistochemistry was done using ImmPRESS™ anti-rabbit Ig (Vector Labs) and diaminobenzidine as substrate.

Endothelial cell culture. Mouse aortic endothelial cells were isolated using collagenase type 2 (Worthington Biochem) as previously described (Yao et al. Blood 93, 1612-21 (1999)) and cultured on fibronectin in endothelial serum-free medium (Gibco) supplemented with 20 ng/mL bFBF and 10 ng/mL EGF. Greater than 90% of cells were of endothelial origin as assessed by von Willebrand factor immunostaining (Dako) and diI-Ac-LDL uptake (Biomedical Technologies).

Water permeability measurements. Cells grown on coverglass were loaded with calcein by incubating with calcein-AM (10 μM, 30 min, Molecular Probes). Coverglasses were mounted in a custom perfusion chamber designed for rapid solution exchange without causing cell detachment. For cell membrane osmotic water permeability measurements, solutions were exchanged from 300 to 150 mOsm PBS and the rate of change of calcein fluorescence was monitored as described previously (Solenov et al. Am J Physiol Cell Physiol 286, C426-32 (2004)).

Adhesion/proliferation. Medium was exchanged 4 hours after plating. Adhesion was defined as the percentage of plated cells remaining immediately after medium exchange. For proliferation, the number of cells was estimated on days 1-4. Cell number was determined using a chromogenic assay kit in 96-well plates (Promega) (Steinle et al. J Biol Chem 277, 43830-5 (2002)). The results were independently confirmed by cell counting.

Migration/invasion. A modified Boyden chamber (Corning Costar) containing polycarbonate membrane filter (6.5 mm diameter, 8 μm pores) coated with gelatin (Miao et al. Cancer Res 61, 7830-9 (2001); Troyanovsky et al. J Cell Biol 152, 1247-54 (2001)) was used to study cell migration/invasion. To detect invasion, the upper surface of the filter was also coated with 20 μL Matrigel (0.3 mg/mL). The upper chamber contained cells in DMEM plus 1% fetal bovine serum, whereas the lower chamber contained DMEM plus 10% fetal bovine serum (chemoattractant) or 1% fetal bovine serum (control). Cells were incubated for 6 hours at 37° C. in 5% CO₂ atmosphere. Non-migrated cells were scraped off the upper surface of the membrane with a cotton swab. Migrated cells remaining on the bottom surface were counted after staining with Coomassie blue. Cell counting done by fluorescence microscopy in experiments where fluorescently labeled cells were used.

Cell volume. Cell diameter was measured after trypsinizing cells, suspending in their medium, and photographing at high magnification in a hemacytometer.

Cord formation. In vitro angiogenesis assay was conducted in Matrigel-coated wells as previously described (Miao et al. Cancer Res 61, 7830-9 (2001); Troyanovsky et al. J Cell Biol 152, 1247-54 (2001)). Cord formation was quantified at 24 hours.

In vitro wound healing. Cells were cultured as confluent monolayers, synchronized in 1% fetal bovine serum for 24 h, and wounded by removing a 300-500 μm strip of cells across the well with a standard 200 μL pipette tip (Lee et al. Am J Physiol Cell Physiol 278, C612-8 (2000)). The wounded monolayers were washed twice to remove non-adherent cells. Wound healing was quantified as the average linear speed of the wound edges over 24 h. Wound healing was also quantified using Image J software as the mean percentage of the remaining cell-free area compared with the area of the initial wound. In some experiments, time-lapse photography of the wound edges was performed (121 frames over 4 min or 25 frames over 4 h, or 181 frames over 15 min or 3 hours). For certain studies cells were seeded in 35-mm Petri dishes with 14 mm diameter and glass-bottomed microwell (MatTek) and keep at 37° C. in a humidified atmosphere with 5% CO₂. Lamellipodia “width” was quantified as lamellipodial area divided by lamellipodial length at the wound edge.

Tumor cell growth, size, proliferation and substrate adherence. Cell growth curves were generated after seeding cells in 24-well plates at a density of 5×10³ cells/ml, and counting triplicate wells every 24 hours for 7 days. Cell diameter was determined by photographing cells at high magnification after trypsinizing and suspending in medium. Cell proliferation was measured by ³H-thymidine incorporation. Three days after seeding cells at 10⁵/ml the culture medium were replaced by serum free medium for 24 h, and then pulsed with 1 μCi/ml [³H]thymidine (Amersham) for 6 h at 37° C. Cells were washed in PBS, solubilized, and DNA was precipitated with 10% ice cold trichloroacetic acid for determination of [³H]thymidine radioactivity by scintillation counting, and DNA content by bisbenzimide (Hoechst 33258, Sigma) fluorescence. Results of the proliferation assays are presented as the mean and standard deviation of triplicate cultures. For measurements of cell substrate adherence, flat-bottom 96-well plate were coated overnight at 4° C. with 10 μg/ml fibronectin, 10 μg/ml collagen I, 20 μg/ml laminin-1, or 1% BSA (as control). Wells were washed with PBS and blocked with 1% BSA for 1 h at 37° C. To each well was added 0.1 ml of cell suspension in Opti-MEM (2×10⁵ cells/ml), plates were incubated for 60 min at 37° C. in 5% CO₂, and non-adherent cells were gently washed away using Opti-MEM. Adherent cells were fixed with 1% glutaraldehyde, stained with 0.1% crystal violet, washed with PBS, and quantified by absorbance at 595 nm in a plate reader.

Tumor cell extravasation in mice. Control and AQP1-expressing tumor cells were labelled in vitro with CMFDA or CMRA as described above. Cells labeled with the red and green fluorescent dyes were mixed at a ratio of 1:1, and 2.5×10⁶ cells suspended in PBS were introduced by tail vein injection. The ratio of green-to-red fluorescent cells in the injected suspension was measured by counting in a fluorescence microscope. Lungs were harvested at 10 minor 6 h after injection. The trachea was cannulated with polyethylene PE-50 tubing, and pulmonary artery with PE-90 tubing. After transecting the left atrium, lungs were perfused in situ with PBS followed by 4% paraformaldehyde (in PBS) at constant pressure (25-35 cm H₂O); 0.5 ml of 4% paraformaldehyde was also infused into the airspaces through the tracheal cannula. Lung tissue was sectioned at 5 μm in a cryostat and fluorescent cells in 5 random fields of each slice were counted.

Tumor growth and metastasis in mice. 10⁶ 4T1 cells were injected intravenously by tail vein in BALB/c mice. Mice were sacrificed after 14 days, and lungs were harvested for hemotoxylin/eosin staining and AQP1 immunohistochemistry. The number of tumor colonies in lung was counted, and colony size and the alveolar wall thickness in a 50 μm peritumoral region were measured using Spot software. In some experiments, 2×10⁵ 4T1 or 10⁶ B16F10 cells were injected subcutaneously between the shoulder blades. Tumor length (L) and width (W) were measured with a caliper for estimation of tumor volume as 0.52×L×W² every 3 days for 18 days.

Cell lines. Fisher rat thyroid epithelial cells (FRT) and Chinese Hamster Ovary cells (CHO-K1) were stably transfected with plasmids to express green fluorescent protein (control), AQP1, or AQP4, and used in adhesion, proliferation, migration and wound healing assays as described above (Yang et al. J Biol Chem 271, 4577-80 (1996).). Greater than 95% of cells expressed the respective proteins. B16F10 melanoma cells (ATCC CRL-6457, American Type Culture Collection) were cultured at 37° C. in a humidified atmosphere containing 5% CO₂ with DMEM supplemented with 4 mM L-glutamine, 100 U ml⁻¹ penicillin, 0.1 mg ml⁻¹ streptomycin and 10% fetal bovine serum. 4T1 mammary gland tumor cells (ATCC CRL-2539) were cultured with RPMI 1640 supplemented with 2 nM L-glutamine, 10 mM HEPES, 1 mM sodium pyruvate, 100 U ml⁻¹ penicillin, 0.1 mg ml⁻¹ streptomycin and 10% fetal bovine serum.

Cell transfection. Cells were plated to ˜80% confluence in 12-well plates 24 h before transfection. 2 μg of recombinant plasmid pcDNA3.1-AQP1 or pcDNA3.1-YFP and 4 μg of Lipofectamin 2000 (Invitrogen) were added into 100 μl Opti-MEM medium for 5 min according to manufacture's instructions. After 48 h the cells were trypsinized and plated on 10-cm diameter dishes, and hygromycin B (500 μg/ml, Roche) was added for selection. At 12-18 days, hygromycin B-resistant cell clones were isolated and transferred to separate culture dishes for expansion and analysis.

Fluorescent labeling. Cells were incubated for 30 min at 37° C. with Opti-MEM containing the fluorescent cell tracker dyes 5-chloromethylfluorescein diacetate (CMFDA, green fluorescent) or a rhodol-based fluorophore (CMRA, red fluorescent) at 2 μM (Molecular Probes). After washing, cells were incubated for an additional 30 min with dye-free medium, washed, and trypsinized.

Statistical analysis. Statistical analysis was done using two-tailed indirect Student t-test, or ANOVA with post-hoc Student-Newman-Keuls and Log-rank test. WinStat (RF Software) and XLStat (AddinSoft) software were used.

### Example 1

### Disruption of AQP1 Function Results in Inhibition of Tumor Growth

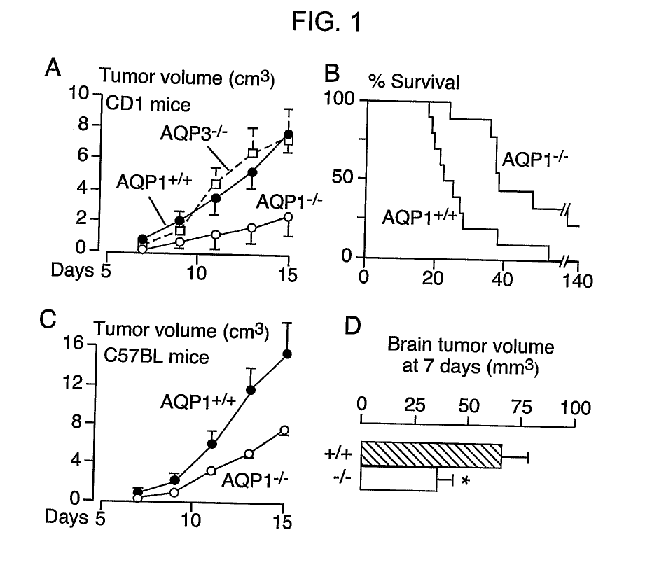

Tumor angiogenesis was studied in a well-established mouse model (Egami et al. J Clin Invest 112, 67-75 (2003); Miao et al. Cancer Res 61, 7830-9 (2001). Melanoma cells were implanted subcutaneously in wildtype (AQP1^{+/+}) and AQP1 null mice (AQP1^{-/-}). In an outbred genetic strain of mice (CD1), tumor growth was greatly slowed in the AQP1 deficient mice (FIG. 1, Panel A). The slowed tumor growth was associated with enhanced survival in the AQP1 deficient mice (AQP1^{-/-}), with 2-fold improvement in median survival and long-term survival of some mice (FIG. 1, Panel B). In control studies, tumor growth was unaffected in mice lacking AQP3 (AQP3^{-/-}) (FIG. 1, Panel A), a water channel that impairs urinary concentrating ability to an even greater extent than AQP1 (Ma et al. Proc Natl Acad Sci USA 97, 4386-91 (2000)). Slow growth of subcutaneous melanoma was also found in syngeneic mice (C57/BL6) lacking AQP1 (FIG. 1, Panel C), and of a brain tumor as assessed histologically at 7 days after intracranial melanoma cell implantation (FIG. 1, Panel D). Thus, the results show that the ability of AQP1 deficiency to limit tumor growth is independent of mouse strain, tumor location, and urinary concentrating ability.

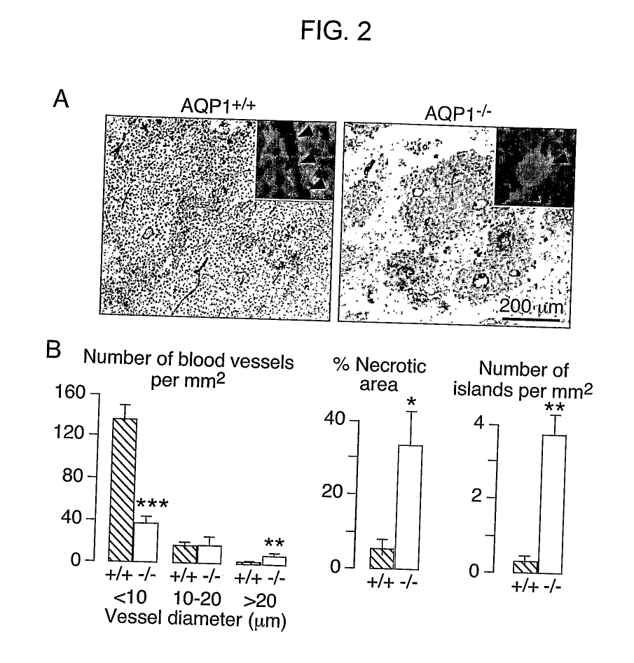

A consistent histological finding in tumors of AQP1 null mice was a dramatically lower density of microvessels and the presence of islands of viable tumor cells surrounded by necrotic tissue (FIG. 2, Panel B). All vessels in tumors of wildtype mice showed strong immunostaining for AQP1 (FIG. 2, Panel A), however, no staining was seen in tumor cells or vessels of AQP1 null mice (FIG. 2, Panel A, inserts). The results show that deletion of AQP1 impairs tumor microvessel proliferation, which leads to extensive tumor necrosis.

### Example 2

### Disruption of AQP1 Function Results in Impaired Angiogenesis

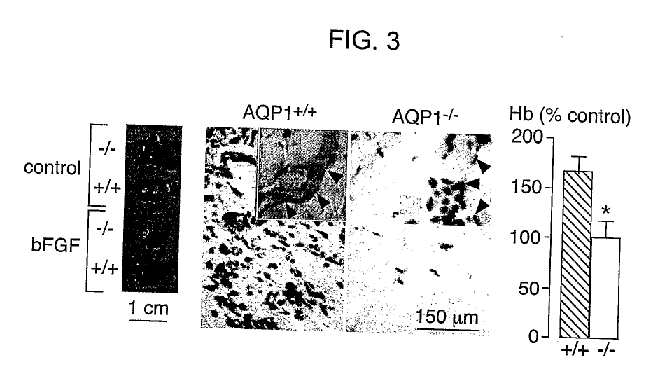

To further confirm the finding that deletion of AQP1 impairs tumor microvessel proliferation, angiogenesis was studied in a tumor-independent model that involves subcutaneous implantation of Matrigel containing the angiogenic factor bFGF (Yao et al. Blood 93, 1612-21 (1999)). The Matrigel mass was removed after 5 days. On gross examination little vascularity was seen in control Matrigel specimens not containing growth factor, whereas Matrigel specimens containing bFGF were reddish-brown with greater staining of specimens from wildtype mice (FIG. 3, left panel). Histological examination revealed substantially more vessel-like structures in the bFCF-supplemented Matrigels from wildtype mice (FIG. 3, middle panel). Total Matrigel hemoglobin, a measure of intact vessel formation (Yao (1999)), was also increased significantly in bFGF-containing Matrigels from wildtype vs. AQP1 null mice (FIG. 3, right panel). The results of the Matrigel experiment show that disruption of AQP1 results in impaired angiogenesis.

### Example 3

### Disruption of AQP1 Function Results in Impaired Cell Migration

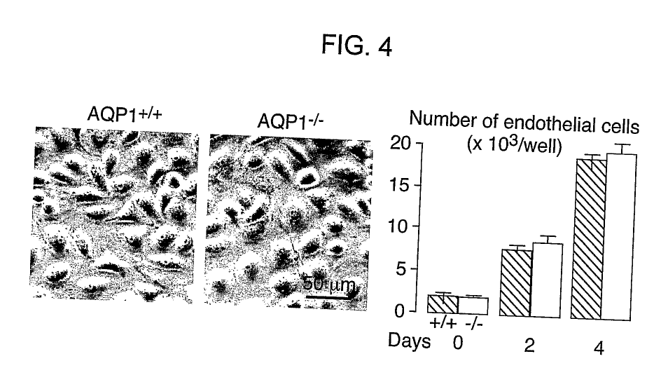

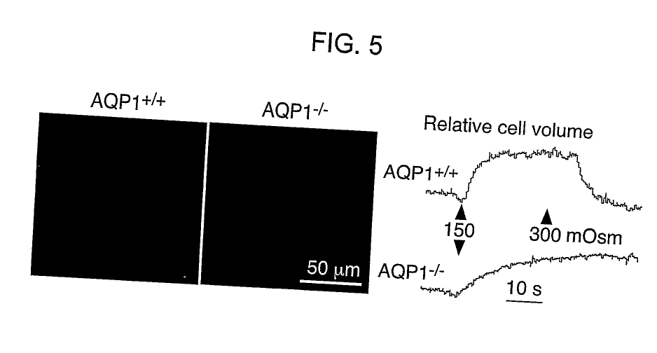

Based on the results from the tumor and Matrigel studies, the effect of disruption of AQP1 on angiogenesis was next examined. It was hypothesized that an intrinsic difference in AQP1-expressing endothelial cells might account for the impaired angiogenesis in AQP1 deficiency. To test this hypothesis, intrinsic endothelial cell functions required for angiogenesis, such as proliferation, adhesion and migration, were studied using cultured endothelial cells. Endothelial cells were generated from mouse aorta (Yao (1999)) and were used 7-10 days after plating. Cells from wildtype (AQP1^{+/+}) and AQP1 null mice (AQP1^{-/-}) had similar appearance by phase-contrast microscopy (FIG. 4, left panel), and growth as determined in a standard cell proliferation assay (FIG. 4, right panel). Greater than 90% of cells from wildtype mice stained positive for AQP1, however, cells from AQP1 null mice showed no staining for AQP1 (FIG. 5, left panel). Functional analysis of osmotic water permeability using a calcein fluorescence quenching method (Solenov et al. Am J Physiol Cell Physiol 286, C426-32 (2004)) revealed a 2.5-fold greater water permeability in the AQP1-expressing endothelial cells as compared to cells AQP1 deficient cells (FIG. 5, right panel; n=4 each, p<0.01).

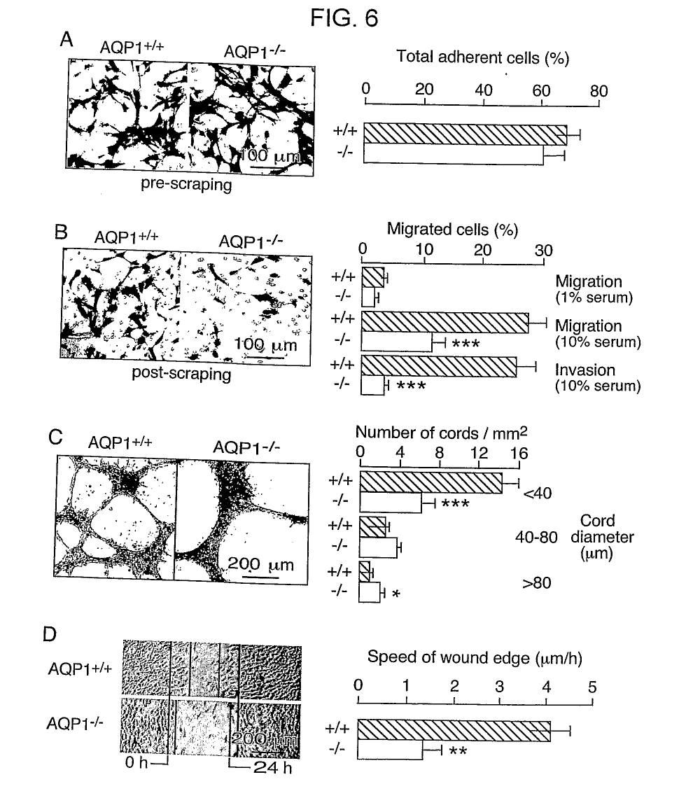

The control vs. AQP1 deficient endothelial cells were used in comparative measurements of cell adhesion, migration, invasion, and cord formation, assayed according to established procedures (Miao et al. Cancer Res 61, 7830-9 (2001); Steinle et al. J Biol Chem 277, 43830-5 (2002); Shi et al., Circ Res 92, 493-500 (2003); Troyanovsky et al. J Cell Biol 152, 1247-54 (2001)). Cell adhesion, as quantified from the number of cells adhering to a gelatin support within 4 hours of plating, was not significantly altered by AQP1 expression (FIG. 6, Panel A). Cell migration towards fetal bovine serum, a potent chemotactic stimulus (Ishida et al. J. Biol. Chem. 278, 34598-604 (2003); Orr et al. J. Cell. Sci. 116, 2917-27 (2003)), was quantified using a modified Boyden chamber. Little migration was seen towards 1% serum, and substantial migration for 10% serum (FIG. 6, Panel B). Migration of AQP1-deficient endothelial cells towards 10% serum was reduced significantly. Even larger differences were found in a cell ‘invasion’ assay in which cells migrated through a Matrigel layer followed by the porous filter. The slower migration of AQP1 deficient vs. wildtype cells was not due to altered cell size (diameter 15.8±0.3 vs. 15.8±0.4 μm, n=100 each). In a study of cord formation, a multi-step process that includes migration, endothelial cells formed nascent cord/tube-like structures within 3 hours. There was a significant reduction in the number of small-diameter cords in AQP1 deficiency at 24 hours and an increase in the number of large-diameter structures (FIG. 6, Panel C), similar to the findings in tumors. The total length of cords was also reduced in AQP1 null cultures (1.9±0.2 vs. 2.9±0.1 mm/mm², n=11 vs. 17, p<0.001). As an independent test of cell migration, wound healing assay were established (Lee et al. Am J Physiol Cell Physiol 278, C612-8 (2000)) in which closure was followed after removal of a 300-500 μm wide strip of cells on a confluent cell monolayer. Wound closure was significantly slowed in AQP1 deficient samples as seen in monolayers photographed at 24 hours after stripping (FIG. 6, Panel D, left panel) and quantified as closure speed (FIG. 6, Panel D, right panel). These results show involvement of AQP1 in endothelial cell migration, offering a mechanism for impaired angiogenesis in AQP1 deficiency in vivo.

### Example 4

### Migration of Non-Endothelial Cells

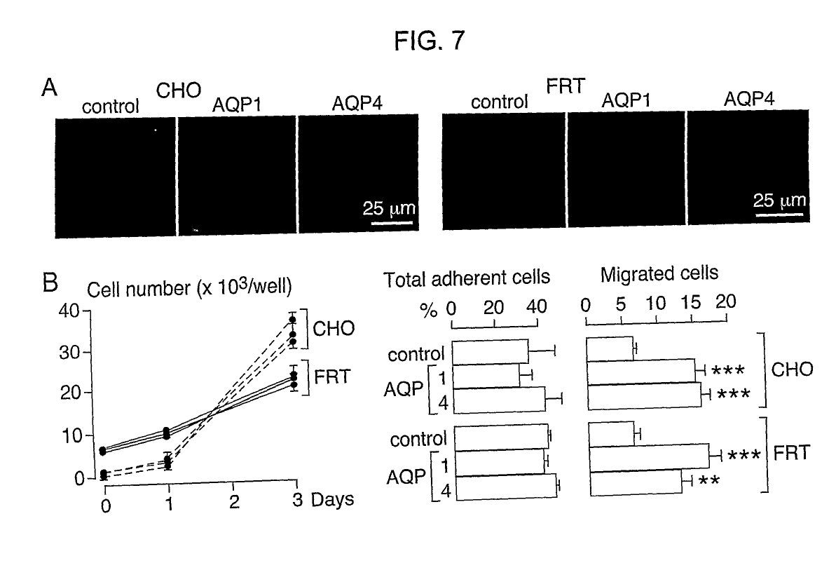

Cell migration involves transient formation of membrane protrusions (lamellipodia, cell membrane ruffles) at the leading edge of the cell that are thought to require rapid local changes in ion fluxes and cell volume (Schwab et al. Pflugers Arch 438, 330-7 (1999); Schwab et al. Am J Physiol Renal Physiol 280, F739-47 (2001); Rosengren et al. Am J Physiol 267, C1623-32 (1994); Lauffenburger et al. Cell 84, 359-69 (1996)), most likely accompanied by fast transmembrane water movement (Condeelis et al. Annu Rev Cell Biol 9, 411-44 (1993)). If transmembrane water movement is a fundamental determinant of cell motility, it can be predicted that a different water channel would also accelerate cell migration, and that the water permeability-dependent rate of migration would be a general phenomenon seen in many cell types. To test this hypothesis, wound healing and cell migration were studied in non-endothelial cells —CHO cells and FRT epithelial cells after stable transfection with control plasmid (encoding green fluorescent protein), or plasmids encoding AQP1, or a structurally different water-selective transporter, AQP4 (Yang et al. J Biol Chem 271, 4577-80 (1996)). Plasma membrane expression of AQP1 or AQP4 were confirmed in the transfected cells (FIG. 7, Panel A, CHO (left) FRT (right)), and osmotic water permeability was increased 6-8 fold compared to their respective control cells. Cell growth and adhesion (FIG. 7, Panel B, left and middle) were not affected by aquaporin expression, whereas cell migration (FIG. 7, Panel B, right) through a porous filter was remarkably enhanced in both cell types when either AQP1 or AQP4 was expressed.

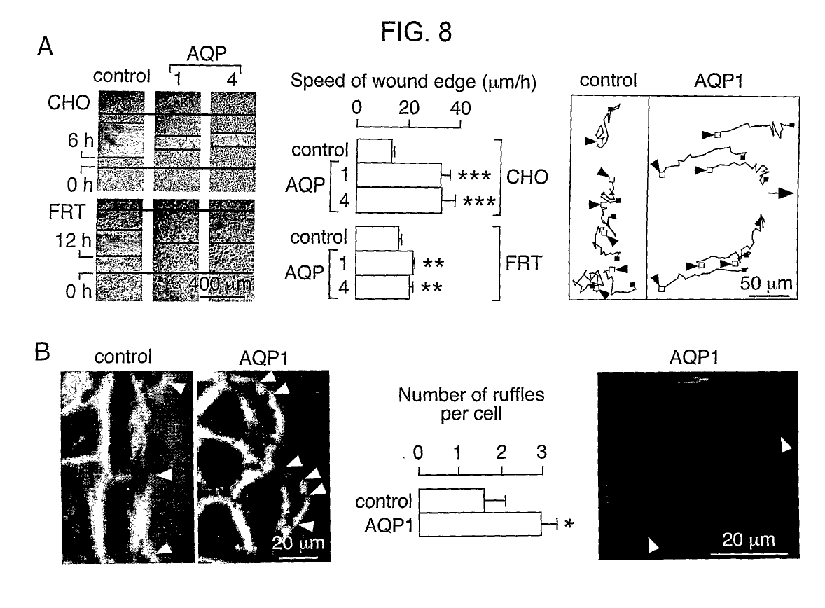

Wound closure experiments supported the conclusion of increased migration in the aquaporin-expressing cells. FIG. 8, Panel A (left and middle) shows significantly accelerated closure in CHO and FRT cells expressing AQP1 or AQP4. FIG. 8, Panel A (right) tracks the movement over 4 hours of six cells at the edge of the wound (arrow indicates starting position) in control and AQP1-expressing CHO cells. Substantially greater directed motion was observed in each of the AQP1-expressing cells, accounting for the accelerated wound healing. In addition, ˜60% of the cells at the wound edge had polarized expression of AQP1 at the leading edge of the cell membrane (FIG. 8, Panel B, left), where water transport is likely to occur. Polarization at the leading edge of migrating cells has been found for several transporters involved in migration, including Na⁺/H⁺ and C₁⁻/HCO₃⁻ exchangers, and the Na⁺/HCO₃⁻ cotransporter (Schwab (2001)).

It was next determined whether polarization of AQP1 might alter the rate of formation of cell membrane protrusions. Time-lapse phase-contrast microscopy was done to quantify the dynamics of cell membrane protrusions in CHO cells at the border of the wound. AQP1 expression produced more protrusions (FIG. 8, Panel B, middle and right) and shorter mean residence time of protrusions (14±1 vs. 36±4 s, 8 cells analyzed per group, p<0.001). Together, these results show that aquaporins accelerate cell migration by facilitating the rapid turnover of cell membrane protrusions at the leading edge.

It has been proposed that actin cleavage and ion uptake at the tip of a lamellipodium create local osmotic gradients that drive the influx of water across the cell membrane (Schwab (2001); Lauffenburger (1996); Condeelis (1993)). Water entry then increases local hydrostatic pressure to cause cell membrane protrusion, which may create space for actin polymerization. These results show that aquaporins provide a pathway for water entry into lamellipodia, though alternative mechanisms may also contribute to aquaporin-dependent cell migration, such as aquaporin interactions with cytoskeletal elements or ion transporters.

### Example 5

### Characterization of Tumor Cell Lines

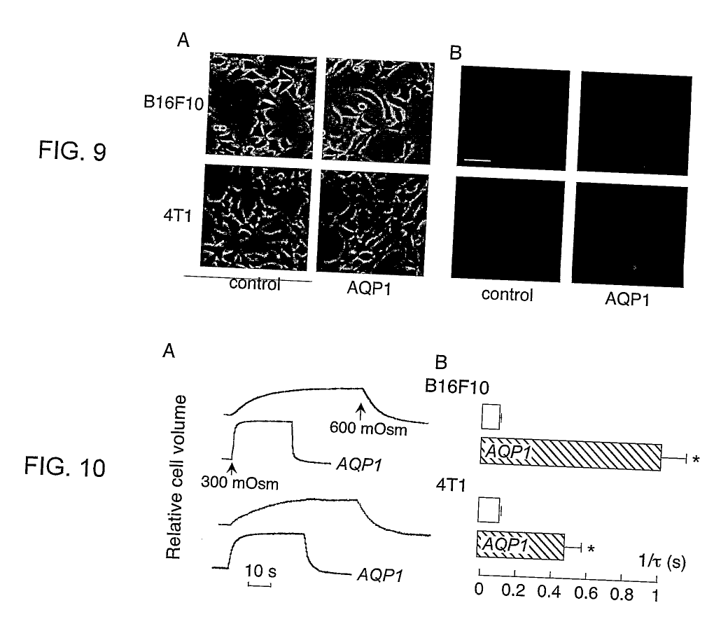

Two tumor cell lines were selected (B16F10 and 4T1 cells) were selected for evaluation of their low water permeability, stable AQP1 expression after transfection, and metastatic potential in mice. Control and AQP1-transfected B16F10 and 4T1 cell lines were characterized. Phase-contrast micrographs in FIG. 9, panel A, show a similar appearance of control and AQP1-expressing cells in each line. FIG. 9, panel B, shows plasma membrane AQP1 protein expression in the transfected cells by immunofluorescence. Osmotic water permeability was measured to verify functional plasma membrane AQP1 expression using a calcein swelling assay (Solenov et al., Am. J. Physiol. 286, C426-432), in which the cytoplasm was stained with calcein prior to inducing osmotic volume changes by superfusion with solutions of different osmolalities. Cell swelling produces an immediate increase in calcein fluorescence upon dilution of cytoplasmic proteins that quench calcein fluorescence. FIG. 10, panel A, shows relatively slow osmotic equilibration in control cells with reciprocal exponential time constant τ⁻¹˜0.1 s⁻¹. Water permeability in the AQP1-transfected cells was substantially increased, consistent with the immunofluorescence data in FIG. 10, panel B.

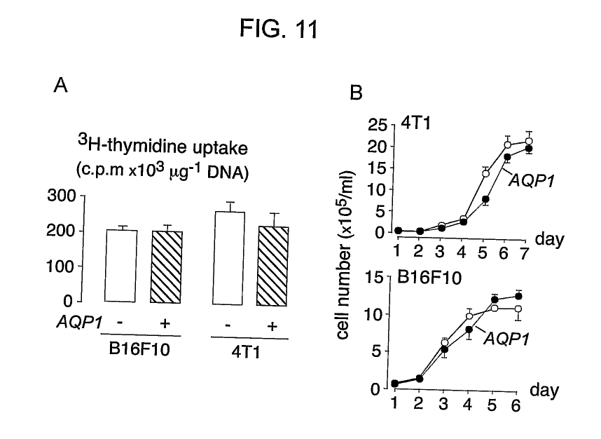

FIG. 11, panels A and B, summarizes proliferation and growth studies and shows that AQP1 expression did not significantly affect the proliferation or growth of B16F10 or 4T1 cells as assayed by ³H-thymidine incorporation and cell counting.

### Example 6

### Increased In Vitro Migration of AQP1-Expressing Tumor Cells

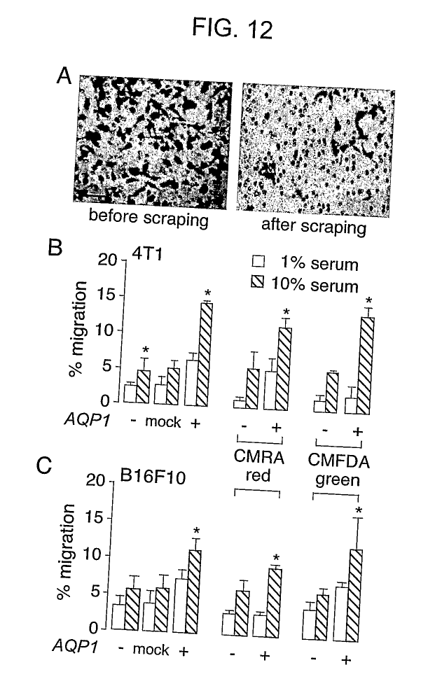

In vitro analysis of tumor cell migration was done using transwell migration and wound healing assays. In the transwell migration assay, cells in medium containing 1% serum were added to the upper surface of a Boyden chamber and allowed to adhere. Migration was measured over 6 hours by contacting the chamber (containing a porous membrane with 8 μm diameter pores) with medium containing 1% (control) or 10% serum. Cells were stained with Coomassie blue, the number of adherent cells was counted, and then non-migrated cells were scraped off the upper surface of the porous filter to reveal the migrated cells (FIG. 12, panel A. FIG. 12, panel B, shows significantly greater migration of AQP1-transfected cells (top—4T1 Cells; bottom—B16F10 Cells).

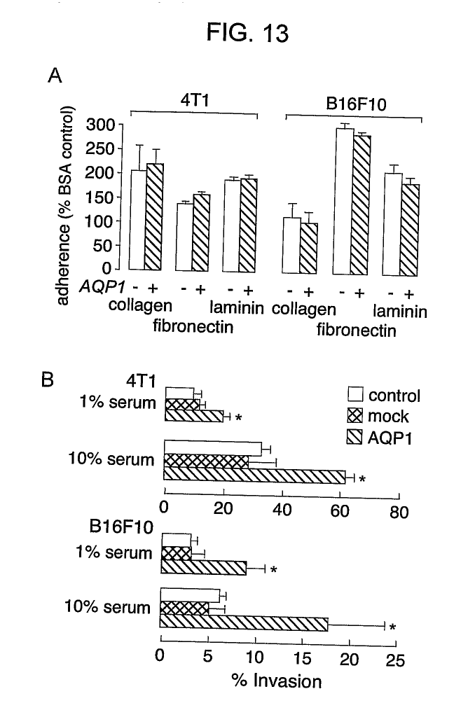

The results show that migration was relatively low in the absence of a chemoattractant stimulus (1% serum) as compared to the presence of a chemoattractant stimulus (10% serum). Cell labeling with the fluorescent cell tracker dyes CMFDA (green fluorescent) or CMRA (red fluorescent), did not significantly affect migration, nor did cell transfection with a YFP-encoding plasmid (“mock”). FIG. 13, panel A, shows that cell adherence to filters coated with collagen, fibronectin or laminin was not different in control vs. AQP1-transfected cells. FIG. 13, panel B, shows that AQP1 expression increased tumor cell migration in an “invasion” assay, in which cells migrated though serial barriers consisting of a Matrigel layer and the porous filter.

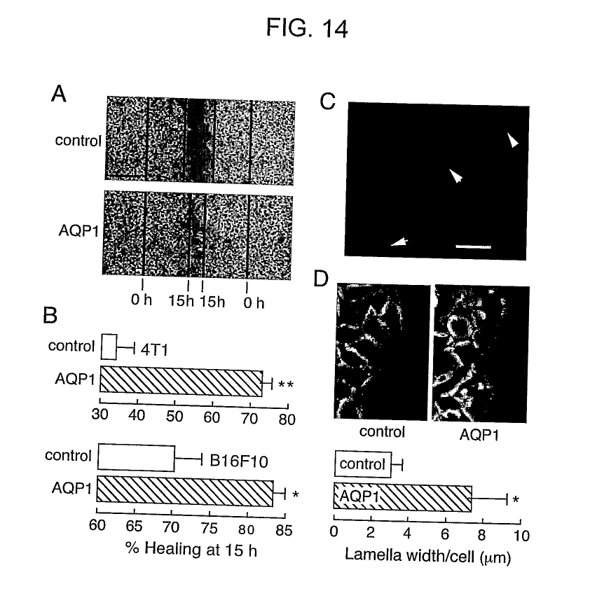

A complementary wound healing assay of in vitro cell migration was done in which a 300 μm-wide linear strip of cells was scraped from confluent monolayers using a pipette tip. Wound closure was quantified from serial micrographs as shown in FIG. 14, panel A. In agreement with the transwell assay, the wound healing assay showed significantly accelerated wound closure in the AQP1-transfected vs. control tumor cells at 15 h after scratch (FIG. 14, panel B). FIG. 14, panel C, shows polarized expression of AQP1 at the leading edge of AQP1-expressing migrating cells at the wound edge, which was seen in 40% of AQP1-expressing B16F10 and 4T1 cells. Interestingly, cell protrusions accompanying migration were quite different in the two cell types, with 4T1 cells showing multiple bleb-like lamellipodia, as seen with CHO cells (Saadoun et al., Nature 434, 786-792), whereas B16F10 cells showed wide lamella (FIG. 14, panel D), as seen in glial cells (Saadoun et al., J Cell Sci. 118, 5691-5698). Video imaging of migrating B16F10 and 4T1 cells showed more frequent bleb/lamella formation after AQP1 transfection, as reported previously for AQP-expressing CHO, glial and proximal tubule cell types (Saadoun et al., Nature 434, 786-792; Solenov et al., Am. J. Physiol. 286, C426-432; (Saadoun et al., J Cell Sci. 118, 5691-5698). AQP1-expressing B16F10 cells showed consistently greater lamellipodial area per leading edge surface (lamellipodial “width”) than control cells (FIG. 14, panel D).

### Example 7

### Increased Extravasation and Metastasis of AQP1-Expressing Tumor Cells In Vivo

Tumor cells were labeled with fixable fluorescent dyes in order to identify them after intravenous injection in mice. Cells were stained with the green fluorescent dye CMFDA or the red fluorescent dye CMRA, each of which is cell permeable and within cells becomes entrapped by covalent reaction with cytoplasmic proteins. Initial studies were done to establish labeling conditions to give bright, stably labeled cells without affecting migration, and to establish a ratio procedure to measure at the same time the migration of control and AQP1-expressing cells. As mentioned above (FIG. 12) the fluorescence labeling did not affect cell migration in vitro. The labeled cells remained fluorescent, with easily distinguishable red vs. green color for greater than 24 hours after labeling.

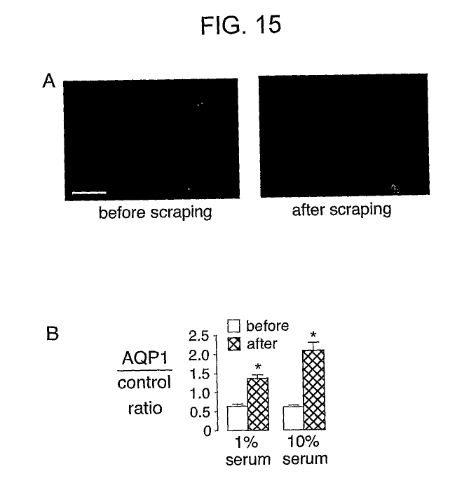

The two-color cell labeling strategy for simultaneous detection of control and AQP1-expressing cells was tested using the in vitro transwell assay in which a mixture of CMRA-labelled control 4T1 cells (red) and CMFDA-labeled AQP1-expressing 4T1 cells (green) was added to the upper chamber. FIG. 15, panel A (left panel), shows the easily recognizable red and green adherent cells at 6 hours after cell addition. More green than red cells remained after scraping the upper surface of the filter (FIG. 15, panel A, right panel). From counting many cells on multiple filters, the ratio of AQP1-expressing vs. control cells was significantly increased after scraping cells (FIG. 15, panel B), in agreement with the data above showing increased migration of AQP1-expressing cells.

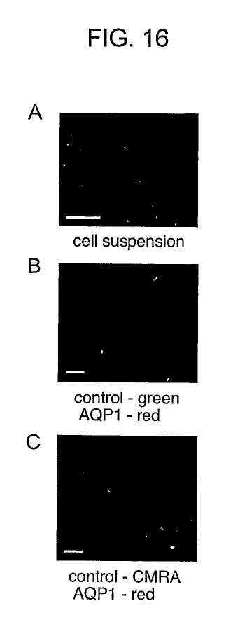

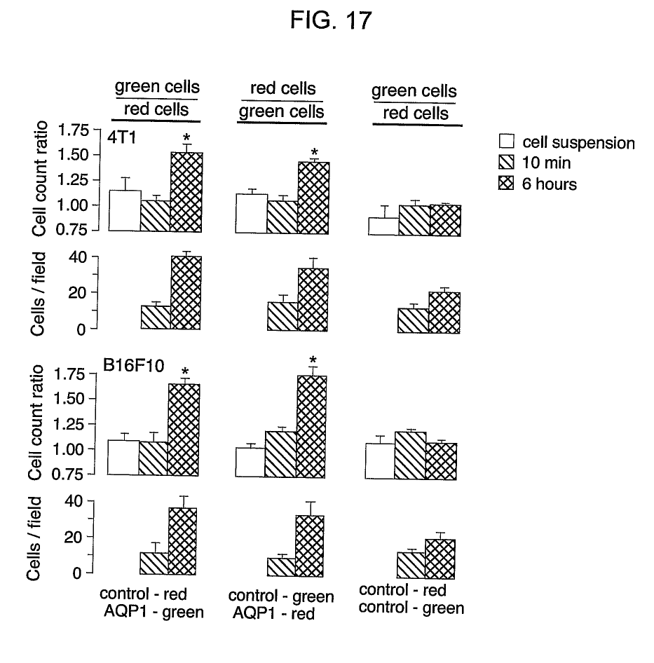

The same approach was used in mice to study tumor cell extravasation, with CMRA labeling of control and CMFDA labeling of AQP1-expressing cells, and with the labeling reversed (CMFDA labeling of control and CMRA labeling of AQP1-expressing cells). FIG. 16, panel A, shows a low magnification fluorescence micrograph of the cell suspension used for tail vein injection, with approximately equal numbers of red- and green-fluorescent cells. However, FIG. 16, panels B and C, show labeled tumor cells in lung at 6 h after tail vein injection, with more AQP1-expressing than control tumor cells for both labeling schemes. The data is summarized in FIG. 17 as cell count ratios and total number of cells per low power microscope field for control and AQP1-expressing 4T1 and B16F10 cells. Migration of AQP1-expressing cells was greater than that of control cells for both labeling schemes at the 6 hour time point (bars labeled 6 hours). In control studies, lungs harvested at 10 min (instead of 6 hours) showed relatively few fluorescent cells, most representing adherent cells to lung endothelium that have not yet migrated. The cell count ratio in lung at 10 min (bars labeled 10 min) was not significantly different from that in the cell suspension used for tail vein injection (bars labeled cell suspension). When a mixture of red- and green-labeled control cells was used for tail vein injections (right panels) the cell count ratio at 6 hours was similar to that at 10 min and in the original cell suspension, as expected. This data provides evidence for increased lung extravasation of AQP1-expressing tumor cells after tail vein injection.

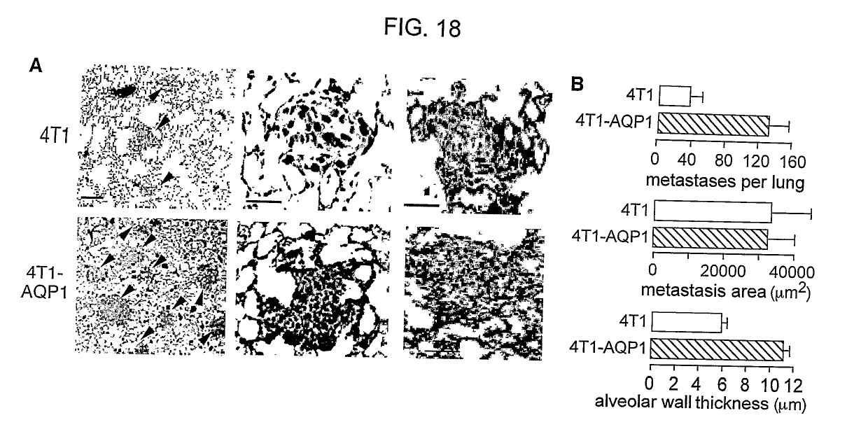

Tumor cell metastasis and growth were also studied in vivo. In one set of experiments lung metastasis was evaluated at 15 days after tail vein injection of control or AQP1-expressing 4T1 cells. FIG. 18, panel A (left panels), shows a greater number of well-demarcated tumor metastases in lungs of mice injected with AQP1-expressing tumor cells. Also, in most mice receiving the AQP1-expressing cells there was evidence for tumor infiltration of alveolar walls (FIG. 18, panel A, middle panels). Immunocytochemistry in FIG. 18, panel A (right panels), shows AQP1 expression in alveoli (microvascular endothelia), as expected, with AQP1 expression in the 4T1-AQP1 cells but not in control 4T1 cells. The data summary in FIG. 18, panel B, shows that AQP1-expressing tumor cells produced a remarkably greater number of lung metastases, but without altered tumor colony size. Alveolar wall thickness within 50 μm of metastases was significantly greater for the AQP1-expressing tumor cells due to tumor cell invasion.

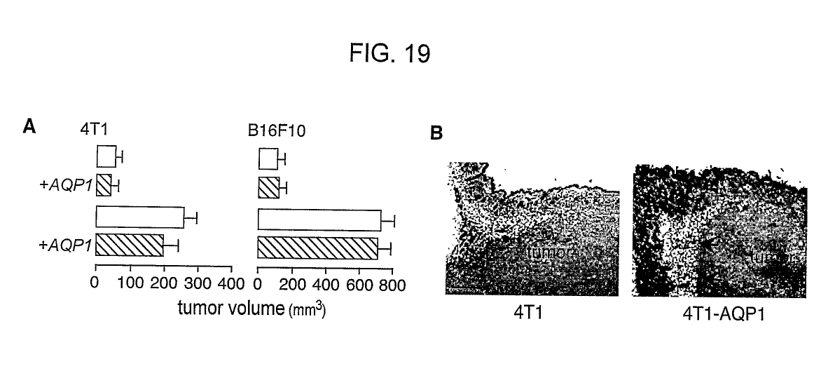

In another set of experiments mice were implanted subcutaneously with control or AQP1-expressing 4T1 or B16F10 cells to study tumor growth and local invasion. FIG. 19, panel A, shows that tumor growth, as assessed by tumor volume at different times after implantation, was not affected by AQP1 expression. However, finger-like projections into subcutaneous adipose tissue were seen with AQP1-expressing but not control 4T1 cells (FIG. 19, panel B). Both AQP1-expressing and control B16F10 tumors were well-encapsulated, without evidence of local invasion.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

That which is claim is:

1. A method of modulating angiogenesis in a subject, comprising administering the subject an agent that modulates a biological activity of an aquaporin 1 (AQP1).

2. The method of claim 1, wherein said agent inhibits the biological activity of the APQ1.

3. The method of claim 1, wherein said agent enhances the biological activity of the AQP11.

4. The method of claim 1, wherein the subject is a human.

5. The method of claim 2, wherein said administering provides for inhibition of angiogenesis in a tumor.

6. The method of claim 3, wherein said administering provides for enhancement of wound healing.

7. A method for treating a cellular proliferative disease in a subject, comprising administering the subject an agent that modulates a biological activity of an aquaporin 1 (AQP1).

8. The method of claim 7, wherein said cellular proliferative disease is cancer.

9. The method of claim 7, wherein said agent inhibits the biological activity of an aquaporin.

10. The method of claim 7, wherein said aquaporin is aquaporin-1.

11. The method of claim 7, wherein the subject is a human.

12. A method of identifying an agent that modulates activity of aquaporin-1, the method comprising,
culturing a cell expressing an aquaporin-1 (AQP1) in the presence of an agent, and
determining the effect of the agent on at least one of cell migration, cell adhesion, or cell proliferation,
wherein a change in one of cell migration, cell adhesion, and cell proliferation in the presence of the agent as compared to the absence of the agent indicates the agent modulates the activity of AQP1.

13. The method of claim 12, wherein the cell is a mammalian cell.

14. The method of claim 12, wherein AQP1 is recombinantly expressed in the cell.

15. The method of claim 12, wherein an increase in one of cell migration, cell adhesion, and cell proliferation, indicates the agent increases activity of the aquaporin.

16. The method of claim 12, wherein a decrease in one of cell migration, cell adhesion, and cell proliferation, indicates the agent increases activity of the aquaporin.

17. The method of claim 12, wherein when said change is inhibition in one of cell migration, cell adhesion, and cell proliferation in the presence of the agent as compared to the absence of the agent indicates the agent is an inhibitor of tumor cell metastasis.

18. A method of identifying an agent that inhibits tumor cell metastasis, the method comprising,
culturing a tumor cell expressing an aquaporin-1 (AQP1) in the presence of an agent, and
determining the effect of the agent on at least one of cell migration, cell adhesion, or cell proliferation,
wherein a change in one of cell migration, cell adhesion, and cell proliferation in the presence of the agent as compared to the absence of the agent indicates the agent is an inhibitor of tumor cell metastasis.

19. The method of claim 18, wherein the cell is a mammalian cell.

20. The method of claim 18, wherein AQP1 is recombinantly expressed in the cell.

21. A pharmaceutical composition comprising a compound of formula (I): 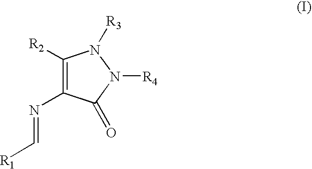
wherein R₁ is independently selected from a substituted or unsubstituted phenyl group; R₂ is independently chosen from a hydrogen, or an alkyl group; R₃ is independently chosen from a hydrogen, or an alkyl group; and R₄ is independently selected from a substituted or unsubstituted phenyl group; or a pharmaceutically acceptable derivative thereof, as an individual stereoisomer or a mixture thereof.

22. The pharmaceutical composition of claim 21, wherein the composition further comprises at least one of a pharmaceutically acceptable carrier, a pharmaceutically acceptable diluent, a pharmaceutically acceptable excipient and a pharmaceutically acceptable adjuvant.

23. The pharmaceutical composition of claim 21, wherein R₁ is a 2-(nitro)-4-(bromo)-5-(hydroxy)phenyl group.

24. The pharmaceutical composition of claim 21, wherein R₂ is a methyl group.

25. The pharmaceutical composition of claim 21, wherein R₃ is a methyl group.

26. The pharmaceutical composition of claim 21, wherein R₄ is an unsubstituted phenyl group.

27. The pharmaceutical compound of claim 26, wherein the compound is:

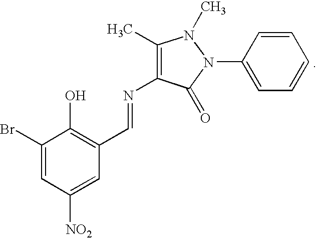

28. A pharmaceutical composition comprising a compound of formula (II): 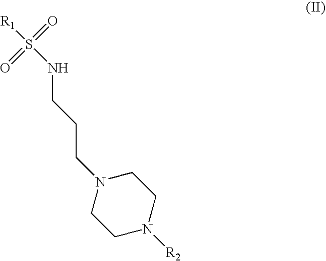
wherein R₁ is independently selected from a substituted or unsubstituted phenyl group, or a substituted or unsubstituted heteroaromatic group; and R₂ is independently selected from a substituted or unsubstituted phenyl group; or a pharmaceutically acceptable derivative thereof, as an individual stereoisomer or a mixture thereof.

29. The pharmaceutical composition of claim 28, wherein the composition further comprises at least one of a pharmaceutically acceptable carrier, a pharmaceutically acceptable diluent, a pharmaceutically acceptable excipient and a pharmaceutically acceptable adjuvant.

30. The pharmaceutical composition of claim 28, wherein R₁ is an unsubstituted quinolinyl group.

31. The pharmaceutical composition of claim 28, wherein R₂ is a 2-(fluoro)phenyl group.

32. The pharmaceutical compound of claim 28, wherein the compound is: [chemical structure]
